(19) United States
(12) Patent Application Publication
(10) Pub. No.: US 2026/0209255 A1
(43) Pub. Date: Jul. 23, 2026

(54) DUAL LIPID STRUCTURES

(71) Applicant: Turn Biotechnologies, Inc., Mountain View, CA (US)

(72) Inventors: Mouhannad JUMAA, Mountain View, CA (US); Zeeshan KAMAL, Mountain View, CA (US); Dipti BARMAN, Mountain View, CA (US)

(21) Appl. No.: 19/141,443

(22) PCT Filed: Dec. 21, 2023
(86) PCT No.: PCT/US2023/085469

§ 371 Date: Jun. 20, 2025

Related U.S. Application Data
(60) Provisional application No. 63/476,977, filed on Dec. 23, 2022.

(51) Int. Cl. C07F 9/10; A61K 9/127; A61K 31/7105; A61K 47/28

(52) U.S. Cl. CPC C07F 9/10; A61K 9/127; A61K 31/7105; A61K 47/28

(57) ABSTRACT

A compound having the following structure of Formula (I): (I) or a stereoisomer, salt, or tautomer thereof, wherein R¹, R², R³, R⁴, R⁵, R⁶, G¹, G², x, y, n, z, and w are as defined herein. Compositions comprising the compounds, and their use in methods of treating diseases, are also described.

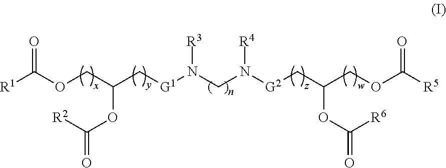

### CROSS-REFERENCES TO RELATED APPLICATIONS

This application claims priority to U.S. provisional patent application No. 63/476,977 filed on Dec. 23, 2022, the content of which is incorporated by reference herein in its entirety.

### TECHNICAL FIELD

The disclosure relates to dual lipids that can be used alone or in combination with additional lipid components, such as helper lipids, stabilization lipids and structural lipids. The disclosure also provides lipid-nanoparticle compositions comprising such lipids for delivery of molecules, particularly nucleic acids.

### BACKGROUND

Nucleic acids-based therapies have attracted attention in the recent years as there is an enormous potential to treat diseases by targeting their genetic blueprints in vivo. Nucleic acid-based therapeutics can achieve long-lasting or even curative effects via gene inhibition, addition, replacement or editing. However, their clinical translation of both nucleic acid medicines and other therapeutic molecules depends on delivery technologies that improve stability, facilitate internalization and/or increase target affinity.

A lipid-based delivery system such as, but not limited to, lipid nanoparticles (LNP), can offer an approach to stabilize and deliver nucleic acids and other therapeutic molecules, and there remains a significant need towards evolution of this technology. Design features, such as optimal particle size, encapsulation efficiencies, robust manufacturing process, different lipophilicity and neutral surface charge, can be further advanced to provide efficient lipid-based delivery systems for nucleic acids and other therapeutic molecules.

### BRIEF SUMMARY

In brief, the present disclosure provides compounds, including stereoisomers, pharmaceutically acceptable salts, or tautomers thereof, which can be used alone or in combination with other therapeutic agents.

In one embodiment, a compound having a structure of Formula (I) is provided:

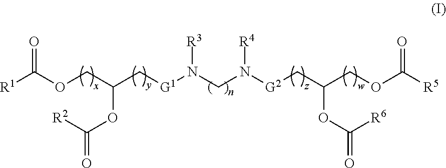

or a stereoisomer, salt, or tautomer thereof, R¹, R², R³, R⁴, R⁵, R⁶, G¹, G², x, y, n, z, and w are as defined herein.

Pharmaceutical compositions comprising one or more of the foregoing compounds of Formula (I) and a therapeutic agent are also provided.

In other embodiments, methods of treatment by administering the foregoing compounds of Formula (I) or the pharmaceutical compositions comprising a compound of Formula (I), to a subject in need thereof to treat a disease is provide.

In one embodiment, a lipid nanoparticle comprising one or more of the foregoing compounds of Formula (I) and a therapeutic agent comprising a nucleic acid are also provided.

In one embodiment, a compound having a structure of Formula (II) is provided:

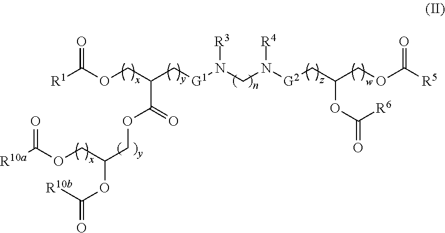

or a stereoisomer, salt, or tautomer thereof, R¹, R^{10a}, R^{10b}, R³, R⁴, R⁵, R⁶, G¹, G², x, y, n, z, and w are as defined herein.

Pharmaceutical compositions comprising one or more of the foregoing compounds of Formula (II) and a therapeutic agent are also provided.

In other embodiments, methods of treatment by administering the foregoing compounds of Formula (II) or the pharmaceutical compositions comprising a compound of Formula (II), to a subject in need thereof to treat a disease is provide.

In one embodiment, a lipid nanoparticle comprising one or more of the foregoing compounds of Formula (II) and a therapeutic agent comprising a nucleic acid are also provided.

Various aspects and embodiments now will be described more fully hereinafter. Such aspects and embodiments make take many different forms and the exemplary ones disclosed herein should not be construed as limiting; rather, these embodiments are provided so that this disclosure will be thorough and complete, and will fully convey its scope to those skilled in the art.

### DETAILED DESCRIPTION

### I. Definitions

For convenience, certain terms employed in the specification and claims are collected here. Unless defined otherwise, all technical and scientific terms used in this disclosure have the same meanings as commonly understood by one of ordinary skill in the art to which this disclosure belongs.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The term “about”, particularly in reference to a given quantity, is meant to encompass deviations of plus or minus five percent.

The compositions of the present disclosure can comprise, consist essentially of, or consist of, the components disclosed.

All percentages, parts and ratios are based upon the total weight of the compositions and all measurements made are at about 25° C., unless otherwise specified.

As used in the specification and appended claims, unless specified to the contrary, the following terms have the meaning indicated:
“Amino” refers to the —NH₂, —NHR, or —NR₂ radical.
“Cyano” refers to the —CN radical.
“Hydroxyl” refers to the —OH radical.
“Imino” refers to the =NH or =NR substituent.
“Nitro” refers to the —NO₂ radical.
“Oxo” refers to the =O substituent.
“Thio” refers to the =S substituent.
“Trifluoromethyl” refers to the —CF₃ radical.
Hyrazido or hydrazino refers to N—N substituent.
wherein each R is a compatible substituent as described in this disclosure. Where an R group is chiral, isomers are contemplated and included herein.

“Alkyl” refers to a linear, saturated, acyclic, monovalent hydrocarbon radical or branched, saturated, acyclic, monovalent hydrocarbon radical, having from one to twelve carbon atoms, preferably one to eight carbon atoms or one to six carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl and the like. An optionally substituted alkyl radical is an alkyl radical that is optionally substituted, valence permitting, by one, two, three, four, or five substituents independently selected from the group consisting of halo, cyano, nitro, aryl, cycloalkyl, heterocyclyl, heteroaryl, oxo, trimethylsilanyl, —OR′, —OC(O)—R′, —N(R′)₂, —C(O)R″, —C(O)OR′, —C(O)N(R′)₂, —N(R′)C(O)OR′″, —N(R′)C(O)R′″, —N(R′)S(O)ₜR′″ (where t is 1 or 2), —S(O)ₜOR′″ (where t is 1 or 2), —S(O)ₚR′″ (where p is 0, 1, or 2) and —S(O)ₜN(R′)₂ (where t is 1 or 2), where each R′ is independently hydrogen, alkyl, haloalkyl, cycloalkyl, cycloalkylalkyl, aryl, aralkyl, heterocyclyl, or heteroaryl; each R″ is independently hydrogen, cycloalkyl, aryl, heterocyclyl, or heteroaryl; and each R′″ is independently alkyl, haloalkyl, cycloalkyl, aryl, heterocyclyl, or heteroaryl.

“Alkoxy” refers to a radical of the formula —ORₐ where Rₐ is an alkyl radical as defined above containing one to twelve carbon atoms. The alkyl part of the optionally substituted alkoxy radical is optionally substituted as defined above for an alkyl radical.

“Alkoxyalkyl” refers to a radical of the formula —Rₐ—O—R_{b} where Rₐ is alkylene and R_{b} is alkyl as defined above. Alkyl and alkylene parts of the optionally substituted alkoxyalkyl radical are optionally substituted as defined above for an alkyl radical and alkylene chain, respectively.

“Aralkyl” refers to a radical of the formula —Rₐ—R_{b}, where Rₐ is alkylene and R_{b} is aryl as described herein. Alkylene and aryl portions of optionally substituted aralkyl are optionally substituted as described herein for alkylene and aryl, respectively.

“Aryl” refers to an aromatic monocyclic or multicyclic hydrocarbon ring system radical containing from 6 to 18 carbon atoms, where the multicyclic aryl ring system is a bicyclic, tricyclic, or tetracyclic ring system. Aryl radicals include, but are not limited to, groups such as fluorenyl, phenyl and naphthyl. An optionally substituted aryl is an aryl radical that is optionally substituted by one, two, three, four, or five substituents independently selected from the group consisting of alkyl, akenyl, halo, haloalkyl, haloalkenyl, cyano, nitro, aryl, heteroaryl, heteroarylalkyl, —R″—OR′, —R″—OC(O)—R′, —R″—N(R′)₂, —R″—C(O)R′, —R″—C(O)OR′, —R″—C(O)N(R′)₂, —R″—N(R′)C(O)OR′″, —R″—N(R′)C(O)R′″, —R″—N(R′)S(O)ₜR′″ (where t is 1 or 2), —R″—S(O)ₜOR′″ (where t is 1 or 2), —R″—S(O)ₚR′″ (where p is 0, 1, or 2), and —R″—S(O)ₜN(R′)₂ (where t is 1 or 2), where each R′ is independently hydrogen, alkyl, haloalkyl, cycloalkyl, aryl, heterocyclyl, or heteroaryl; each R″ is independently a direct bond or a linear or branched alkylene or alkenylene chain; and each R′″ is independently alkyl, haloalkyl, cycloalkyl, cycloalkylalkyl, aryl, heterocyclyl, or heteroaryl.

“Arylalkoxy” refers to a group of formula —O—R, where R is aralkyl. An optionally substituted arylalkoxy is an arylalkoxy that is optionally substituted as described herein for aralkyl. In some embodiments, arylalkoxy is benzyloxy.

“Cycloalkyl” refers to a stable non-aromatic monocyclic or polycyclic hydrocarbon radical having from three to fifteen carbon atoms, preferably having from three to ten carbon atoms, and which is saturated or unsaturated, and which attaches to the rest of the molecule by a single bond. A polycyclic hydrocarbon radical is bicyclic, tricyclic, or tetracyclic ring system. An unsaturated cycloalkyl contains one, two, or three carbon-carbon double bonds and/or one carbon-carbon triple bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, and the like. An optionally substituted cycloalkyl is a cycloalkyl radical that is optionally substituted by one, two, three, four, or five substituents independently selected from the group consisting of alkyl, alkenyl, halo, haloalkyl, haloalkenyl, cyano, nitro, oxo, aryl, aralkyl, cycloalkyl, heterocyclyl, heteroaryl, —R″—OR′, —R″—OC(O)—R′, —R″—N(R′)₂, —R″—C(O)R′, —R″—C(O)OR′, —R″—C(O)N(R′)₂, —R″—N(R′)C(O)OR′″, —R″—N(R′)C(O)R′″, —R″—N(R′)S(O)ₜR′″ (where t is 1 or 2), —R″—S(O)ₜOR′″ (where t is 1 or 2), —R″—S(O)ₚR′″ (where p is 0, 1, or 2) and —R″—S(O)ₜN(R′)₂ (where t is 1 or 2) where each R′ is independently hydrogen, alkyl, haloalkyl, cycloalkyl, aryl, heterocyclyl, or heteroaryl; each R″ is independently a direct bond or a linear or branched alkylene or alkenylene chain; and each R′″ is independently alkyl, haloalkyl, cycloalkyl, cycloalkylalkyl, aryl, aralkyl, heterocyclyl, or heteroaryl.

“Deuterated compounds” are compounds where one of more hydrogen atoms have been replaced with a deuterium atom. Deuterated drugs may be derivatives of an active compound. Deuterated drugs may be prodrugs. Deuteration may alter the physical properties, metabolic properties, activity or safety of a drug.

“Derivatives” are related chemical species that can be derived from a similar compound via chemical reactions. They may encompass slight chemical modifications, substitution of atoms with deuterated atoms, substitution of atoms with stable or radioactive isotopes or other modifications that imbue a compound with desirable properties.

“Fused” refers to any ring system described herein which is fused to an existing ring structure in the compounds. When the fused ring system is a heterocyclyl or a heteroaryl, any carbon atom on the existing ring structure which becomes part of the fused ring system may be replaced with a nitrogen atom.

“Halo” refers to the halogen substituents: bromo, chloro, fluoro, and iodo.

“Haloalkyl” refers to an alkyl radical, as defined above, that is further substituted by one or more halogen substituents. The number of halo substituents included in haloalkyl is from one and up to the total number of the hydrogen atoms available for replacement with the halo substituents (e.g., perfluoroalkyl). Non-limiting examples of haloalkyl include trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl and the like. For an optionally substituted haloalkyl, the hydrogen atoms bonded to the carbon atoms of the alkyl part of the haloalkyl radical may be optionally replaced with substituents as defined above for an optionally substituted alkyl.

“Haloalkenyl” refers to an alkenyl radical, as defined above, that is further substituted by one or more halo substituents. The number of halo substituents included in haloalkenyl is from one and up to the total number of the hydrogen atoms available for replacement with the halo substituents (e.g., perfluoroalkenyl). Non-limiting examples of haloalkenyl include 2,2-difluoroethenyl, 3-chloroprop-1-enyl, and the like. For an optionally substituted haloalkenyl, the hydrogen atoms bonded to the carbon atoms of the alkenyl part of the haloalkenyl radical may be optionally replaced with substitutents as defined above for an optionally substituted alkenyl group.

“Haloalkynyl” refers to an alkynyl radical, as defined above, that is further substituted by one or more halo substituents. The number of halo substituents included in haloalkynyl is from one and up to the total number of the hydrogen atoms available for replacement with the halo substituents (e.g., perfluoroalkynyl). Non-limiting examples of haloalkynyl include 3-chloroprop-1-ynyl and the like. The alkynyl part of the haloalkynyl radical may be additionally optionally substituted as defined above for an alkynyl group.

“Heteroarylalkyl” refers to a radical of the formula —Rₐ—R_{b}, where Rₐ is alkylene and R_{b} is heteroaryl as described herein. Alkylene and heteroaryl portions of optionally substituted heteroarylalkyl are optionally substituted as described herein for alkylene and heteroaryl, respectively.

“Heterocyclyl” refers to a stable 3- to 18-membered non-aromatic ring system radical having the carbon count of two to twelve and containing a total of one to six heteroatoms independently selected from the group consisting of nitrogen, oxygen, phosphorus, and sulfur. A heterocyclyl radical is a monocyclic, bicyclic, tricyclic, or tetracyclic ring system. A bicyclic, tricyclic, or tetracyclic heterocyclyl is a fused, spiro, and/or bridged ring system. The heterocyclyl radical may be saturated or unsaturated. An unsaturated heterocyclyl contains one, two, or three carbon-carbon double bonds and/or one carbon-carbon triple bond. An optionally substituted heterocyclyl is a heterocyclyl radical that is optionally substituted by one, two, three, four, or five substituents independently selected from the group consisting of alkyl, alkenyl, halo, haloalkyl, haloalkenyl, cyano, oxo, thioxo, nitro, aryl, aralkyl, cycloalkyl, heterocyclyl, heteroaryl, —R″—OR′, —R″—OC(O)—R′, —R″—N(R′)₂, —R″—C(O)R′, —R″—C(O)OR′, —R″—C(O)N(R′)₂, —R″—N(R′)C(O)OR′″, —R″—N(R′)C(O)R′″, —R″—N(R′)S(O)ₜR′″ (where t is 1 or 2), —R″—S(O)ₜOR′″ (where t is 1 or 2), —R″—S(O)ₚR′″ (where p is 0, 1, or 2), and —R″—S(O)ₜN(R′)₂ (where t is 1 or 2), where each R′ is independently hydrogen, alkyl, alkenyl, haloalkyl, cycloalkyl, aryl, heterocyclyl, or heteroaryl; each R″ is independently a direct bond or a linear or branched alkylene or alkenylene chain; and each R′″ is independently alkyl, alkenyl, haloalkyl, cycloalkyl, aryl, heterocyclyl, or heteroaryl. The nitrogen, carbon, or sulfur atoms in the heterocyclyl radical may be optionally oxidized (when the substituent is oxo and is present on the heteroatom); the nitrogen atom may be optionally quaternized (when the substituent is alkyl, alkenyl, aryl, aralkyl, cycloalkyl, heterocyclyl, heteroaryl, —R″—OR′, —R″—OC(O)—R′, —R″—N(R′)₂, —R″—C(O)R′, —R″—C(O)OR′, —R″—C(O)N(R′)₂, —R″—N(R′)C(O)OR′″, —R″—N(R′)C(O)R′″, —R″—N(R′)S(O)ₜR′″ (where t is 1 or 2), —R″—S(O)ₜOR′″ (where t is 1 or 2), —R″—S(O)ₚR′″ (where p is 0, 1, or 2), and —R″—S(O)ₜN(R′)₂ (where t is 1 or 2), where R″ is a linear or branched alkylene or alkenylene chain, and R′ and R′″ are as defined above). Examples of optionally substituted heterocyclyl radicals include, but are not limited to, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

“Heterocyclylene” refers to a heterocyclyl in which one hydrogen atom is replaced with a valency. An optionally substituted heterocyclylene is optionally substituted as described herein for heterocyclyl.

“Heteroaryl” refers to a 5- to 18-membered ring system radical containing at least one aromatic ring, having the carbon count of one to seventeen carbon atoms, and containing a total of one to ten heteroatoms independently selected from the group consisting of nitrogen, oxygen, and sulfur. The heteroaryl radical is a monocyclic, bicyclic, tricyclic, or tetracyclic ring system. The bicyclic, tricyclic, or tetracyclic heteroaryl radical is a fused and/or bridged ring system. An optionally substituted heteroaryl is a heteroaryl radical that is optionally substituted by one, two, three, four, or five substituents independently selected from the group consisting of alkyl, alkenyl, alkoxy, halo, haloalkyl, haloalkenyl, cyano, oxo, thioxo, nitro, oxo, aryl, aralkyl, cycloalkyl, heterocyclyl, heteroaryl, or heteroarylalkyl, —R″—OR′, —R″—OC(O)—R′, —R″—N(R′)₂, —R″—C(O)R′, —R″—C(O)OR′, —R″—C(O)N(R′)₂, —R″—N(R′)C(O)OR′″, —R″—N(R′)C(O)R′″, —R″—N(R′)S(O)ₜR′″ (where t is 1 or 2), —R″—S(O)ₜOR′″ (where t is 1 or 2), —R″—S(O)ₜR′″ (where p is 0, 1, or 2), and —R″—S(O)ₜN(R′)₂ (where t is 1 or 2), where each R′ is independently hydrogen, alkyl, alkenyl, haloalkyl, cycloalkyl, aryl, heterocyclyl, or heteroaryl; each R″ is independently a direct bond or a linear or branched alkylene or alkenylene chain; and each R′″ is alkyl, alkenyl, haloalkyl, cycloalkyl, aryl, heterocyclyl, or heteroaryl. The nitrogen, carbon, or sulfur atoms in the heterocyclyl radical may be optionally oxidized (when the substituent is oxo and is present on the heteroatom), provided that at least one ring in heteroaryl remains aromatic; the nitrogen atom may be optionally quaternized (when the substituent is alkyl, alkenyl, aryl, aralkyl, cycloalkyl, heterocyclyl, heteroaryl, —R″—OR′, —R″—OC(O)—R′, —R″—N(R′)₂, —R″—C(O)R′, —R″—C(O)OR′, —R″—C(O)N(R′)₂, —R″—N(R′)C(O)OR′″, —R″—N(R′)C(O)R′″, —R″—N(R′)S(O)ₜR′″ (where t is 1 or 2), —R″—S(O)ₜOR′″ (where t is 1 or 2), —R″—S(O)ₚR′″ (where p is 0, 1, or 2), and —R″—S(O)ₜN(R′)₂ (where t is 1 or 2), where R″ is a linear or branched alkylene or alkenylene chain, and R′ and R′″ are as defined above), provided that at least one ring in heteroaryl remains aromatic. Examples of optionally substituted heteroaryl radicals include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyl, naphthyridinyl, oxadiazolyl, 2-oxoazepinyl, oxazolyl, oxiranyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrrolyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl and thiophenyl (i.e., thienyl).

The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

“Prodrugs” are compounds that after administration are metabolized or otherwise chemically transformed into an active moiety. Prodrugs may be derivatives of an active compound. Prodrugs may or may not be active prior to conversion into an active form in vivo.

The term “treating” is used herein, for instance, in reference, for example, to methods of treating a condition or disease, and generally includes the administration of a compound or composition which reduces the frequency of, or delays the onset of, symptoms of the condition or disease (e.g., cancer, autoimmune disease, inflammatory disorder, gastrointestinal disorder) in a subject relative to a subject not receiving the compound or composition. This can include reversing, reducing, or arresting the symptoms, clinical signs, and underlying pathology of a condition in a manner to improve or stabilize a subject's condition (e.g., regression of a cancer, of aging of a cell or a tissue, of an autoimmune or inflammatory disease, etc.).

The embodiments disclosed herein encompass all pharmaceutically acceptable compounds of the compound of (I)—(IIB) being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabelled compounds could be useful to help determine or measure the effectiveness of the compounds, by characterizing, for example, the site or mode of action, or binding affinity to pharmacologically important site of action. Certain isotopically-labelled compounds of (I)—(IIB), for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of (I)—(IIB) can generally be prepared by conventional techniques known to those skilled in the art or by processes analogous to those described in the Preparations and Examples as set out below using an appropriate isotopically-labeled reagent in place of the non-labeled reagent previously employed.

The embodiments disclosed herein encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound of this disclosure to a mammal for a period of time sufficient to yield a metabolic product thereof. Such products are typically identified by administering a radiolabelled compound of the disclosure in a detectable dose to an animal, such as rat, mouse, guinea pig, monkey, or to human, allowing sufficient time for metabolism to occur, and isolating its conversion products from the urine, blood or other biological samples.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid (TFA), undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2 dimethylaminoethanol, 2 diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N ethylpiperidine, polyamine resins and the like. Particularly preferred organic bases are isopropylamine, diethylamine, ethanolamine, trimethylamine, dicyclohexylamine, choline and caffeine.

A “pharmaceutical composition” refers to a formulation of a compound of the disclosure and a medium generally accepted in the art for the delivery of the biologically active compound to mammals, e.g., humans. Such a medium includes all pharmaceutically acceptable carriers, diluents and excipients therefor.

“Effective amount” or “therapeutically effective amount” refers to that amount of a compound of the disclosure which, when administered to a mammal, preferably a human, is sufficient to effect treatment in the mammal, preferably a human. The amount of a compound which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposeable mirror images of one another. The present disclosure also contemplates “diastereomers”, which refers to non-mirror image of non-identical stereoisomers. Diastereomers occur when two or more stereoisomers of a compound have different configurations at one or more of the equivalent stereocenters and are not mirror images of each other.

A “tautomer” refers to a proton shift from one atom of a molecule to another atom of the same molecule. The present disclosure includes tautomers of any said compounds.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

### II. Compounds

The compounds described herein are ionizable lipids with degradable head group. In one embodiment, a compound having a structure of Formula (I) is provided:

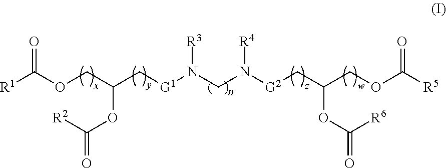

or stereoisomer, salt, or tautomer thereof, wherein G¹ and G² are each independently C₁-C₆ alkyl ester, C₁-C₆ alkyl amide, or C₁-C₆ alkyl phosphate, wherein one of G¹ and G² is C₁-C₆ alkyl amide or C₁-C₆ alkyl phosphate, R¹, R², R⁵, and R⁶ are each independently C₁₂-C₂₄ alkyl, C₁₂-C₂₄ alkylene, or C₁-C₆ alkyl, wherein the C₁-C₆ alkyl is substituted with —OC(═O)R⁷, —NR⁸C(═O)R⁷, or —C(═O)OR⁷; R³ and R⁴ are each independently H, C₁-C₆ alkyl, or C₁-C₆ alkyl alcohol; R⁷ is branched C₆-C₂₄ alkyl or C₂-C₁₆ heteroalkyl; R⁸ is H or C₁-C₆ alkyl; x and w are each independently an integer ranging from 1 to 4; n is an integer ranging from 1 to 12; and y, and z are each independently an integer ranging from 1 to 6.

In one embodiment, G¹ and G² are each independently C₁-C₆ alkyl ester, C₁-C₆ alkyl amide, or C₁-C₆ alkyl phosphate, wherein one of G¹ and G² is C₁-C₆ alkyl amide or C₁-C₆ alkyl phosphate. In some embodiments, G¹ and G² are each independently C₁-C₆ alkyl ester or C₁-C₆ alkyl phosphate. In one embodiment, G¹ and G² are each independently C₁-C₄ alkyl ester or C₁-C₄alkyl phosphate. In one embodiment, G¹ and G² are each independently C₁-C₃ alkyl ester or C₁-C₃ alkyl phosphate. In one embodiment, G¹ and G² are each independently C₃ alkyl ester or C₂ alkyl phosphate.

In one embodiment, the compound has one of the following structures (IA)-(ID):

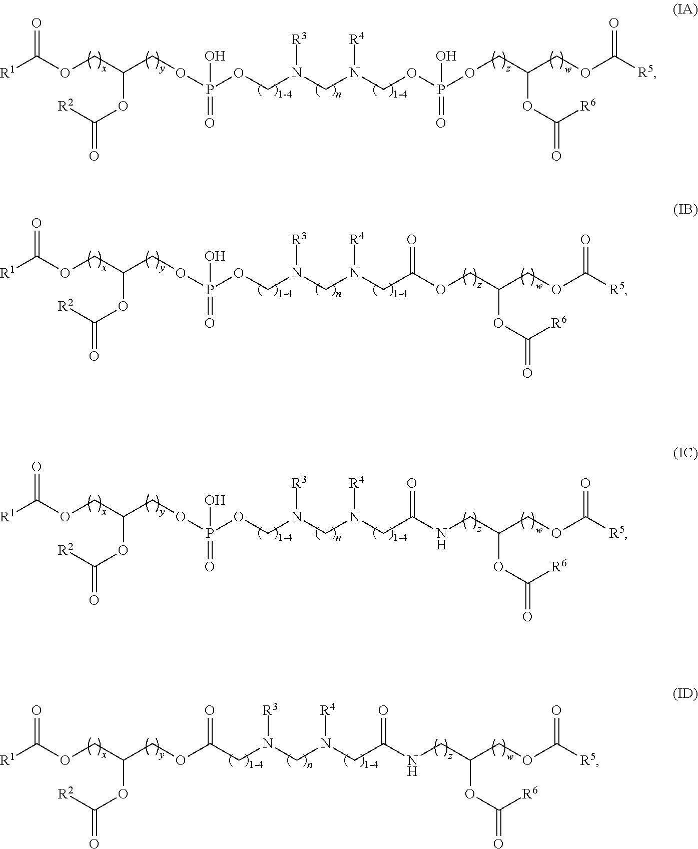

or a pharmaceutically acceptable salt or stereoisomer thereof.

In one embodiment, R¹, R², R⁵, and R⁶ are each independently C₁₂-C₂₄ alkylene or C₁-C₆ alkyl, wherein the C₁-C₆ alkyl is substituted with —OC(═O)R⁷, —NR⁸C(═O)R⁷, or —C(═O)OR⁷. In some embodiments, R¹, R², R⁵, and R⁶ are each independently C₁₂-C₂₄ alkylene or C₁-C₆ alkyl, wherein the C₁-C₆ alkyl is substituted with —OC(═O)R⁷ or —NR⁸C(═O)R⁷. In some embodiments, R¹, R², R⁵, and R⁶ are each independently C₁₂-C₂₄ alkylene or C₁-C₆ alkyl, wherein the C₁-C₆ alkyl is substituted with —OC(═O)R⁷. In some embodiments, R¹, R², R⁵, and R⁶ are each independently C₁₄-C₂₀ alkylene or C₂-C₅ alkyl, wherein the C₂-C₅ alkyl is substituted with —OC(═O)R⁷ or —NR⁸C(═O)R⁷. In some embodiments, R², R⁵, and R⁶ are each independently C₁₄-C₂₀ alkylene or C₂-C₅ alkyl, wherein the C₂-C₅ alkyl is substituted with —OC(═O)R⁷. In some embodiments, R¹, R², R⁵, and R⁶ are each independently C₁₇ alkylene or C₄ alkyl, wherein the C₄ alkyl is substituted with —OC(═O)R⁷. In some embodiments, R¹ and R² are C₁₇ alkylene and R⁵ and R⁶ are each independently C₁₇ alkylene or C₄ alkyl, wherein the C₄ alkyl is substituted with —OC(═O)R⁷.

In one embodiment, R⁷ has one of the following structures:

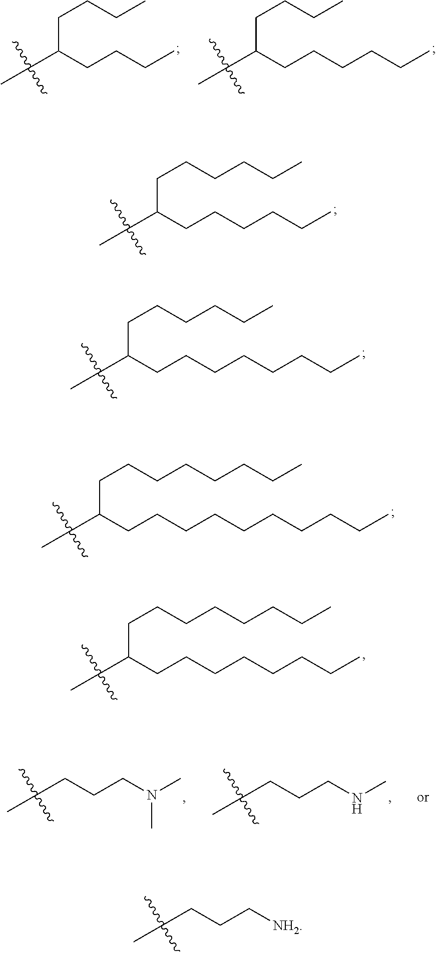

In some embodiments, R⁷ is

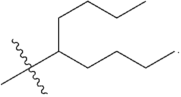 [SMILES: *C(CCCC)CCCC]

In some embodiments, R⁷ is

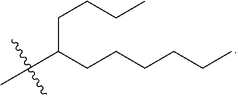 [SMILES: *C(CCCC)CCCCCC]

In some embodiments, R⁷ is

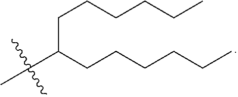 [SMILES: *C(CCCCCC)CCCCCC]

In some embodiments, R⁷ is

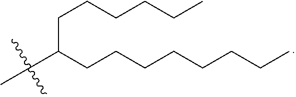 [SMILES: *C(CCCCCC)CCCCCCCC]

In some embodiments, R⁷ is

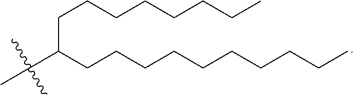 [SMILES: *C(CCCCCCCC)CCCCCCCCCC]

In some embodiments, R⁷ is

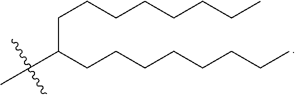 [SMILES: *C(CCCCCCCC)CCCCCCCC]

In some embodiments, R⁷ is

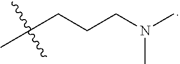 [SMILES: *CCCN(C)C]

In some embodiments, R⁷ is

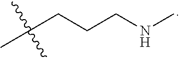 [SMILES: *CCCNC]

In some embodiments, R⁷ is

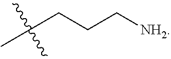 [SMILES: *CCCN]

In one embodiment, R⁷ has one of the following structures:

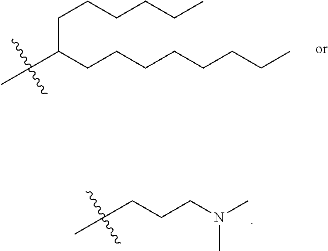

In one embodiment, R⁸ is H or C₁-C₃ alkyl. In some embodiments, R⁸ is H or methyl (—CH₃).

In one embodiment, R¹, R², R⁵, and R⁶ each independently have one of the following structures:

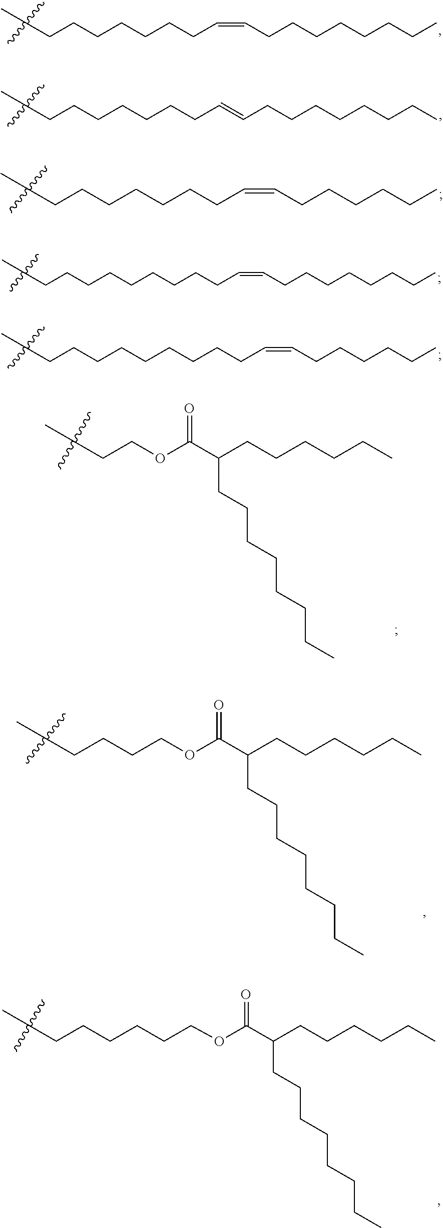

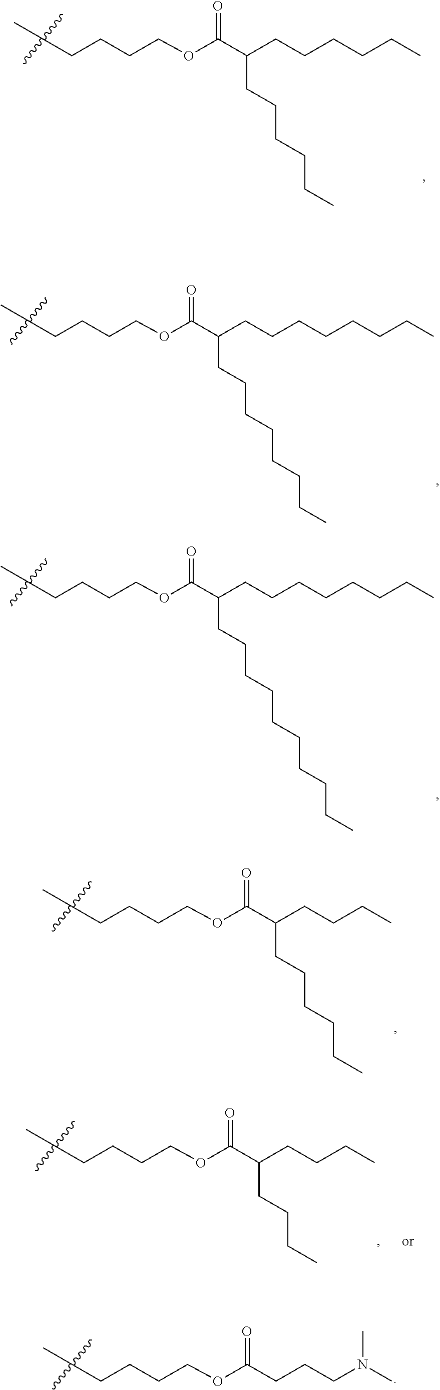 [SMILES: *CCCCOC(=O)C(CCCCCC)CCCCCC ; *CCCCOC(=O)C(CCCCCCCC)CCCCCCCC ; *CCCCOC(=O)C(CCCCCCCC)CCCCCCCCCC ; *CCCCOC(=O)C(CCCC)CCCCCC ; *CCCCOC(=O)C(CCCC)CCCC ; *CCCCOC(=O)CCCN(C)C]

In some embodiments, R¹, R², R⁵, or R⁶ is

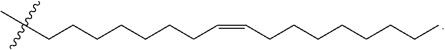 [SMILES: *CCCCCCC/C=C\CCCCCCCC]

In some embodiments, R¹, R², R⁵, or R⁶ is

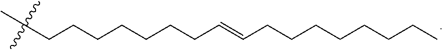 [SMILES: *CCCCCCC/C=C/CCCCCCCC]

In some embodiments, R¹, R², R⁵, or R⁶ is

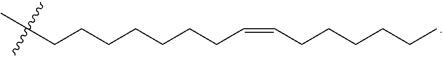 [SMILES: *CCCCCCC/C=C\CCCCCC]

In some embodiments, R¹, R², R⁵, or R⁶ is

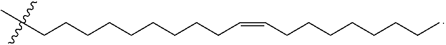 [SMILES: *CCCCCCCCC/C=C\CCCCCCCC]

In some embodiments, R¹, R², R⁵, or R⁶ is

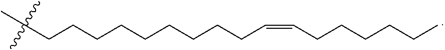 [SMILES: *CCCCCCCCC/C=C\CCCCCC]

In some embodiments, R¹, R², R⁵, or R⁶ is

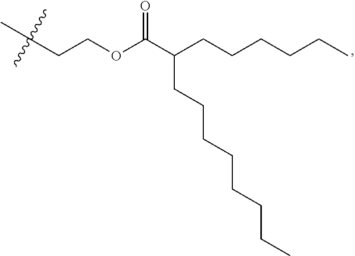 [SMILES: *CCOC(=O)C(CCCCCC)CCCCCCCC]

In some embodiments, R¹, R², R⁵, or R⁶ is

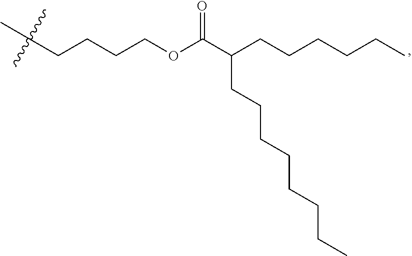 [SMILES: *CCCCOC(=O)C(CCCCCC)CCCCCCCC]

In some embodiments, R¹, R², R⁵, or R⁶ is

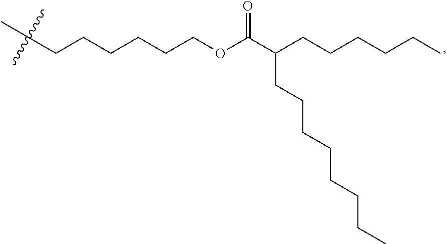 [SMILES: *CCCCCCOC(=O)C(CCCCCC)CCCCCCCC]

In some embodiments, R¹, R², R⁵, or R⁶ is

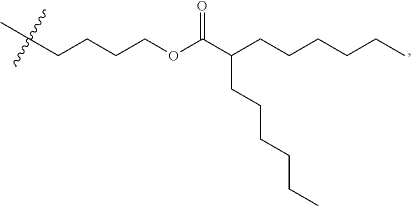 [SMILES: *CCCCOC(=O)C(CCCCCC)CCCCCC]

In some embodiments, R¹, R², R⁵, or R⁶ is

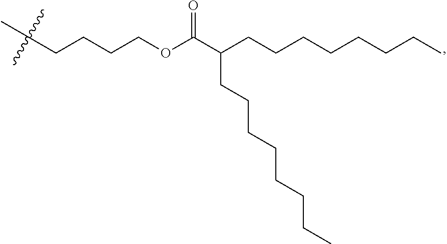 [SMILES: *CCCCOC(=O)C(CCCCCCCC)CCCCCCCC]

In some embodiments, R¹, R², R⁵, or R⁶ is

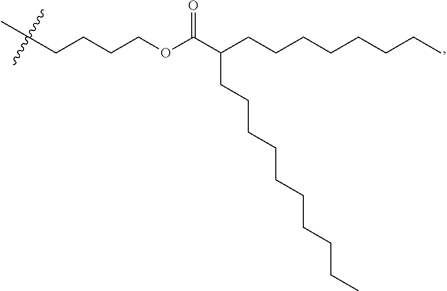 [SMILES: *CCCCOC(=O)C(CCCCCCCC)CCCCCCCCCC]

In some embodiments, R¹, R², R⁵, or R⁶ is

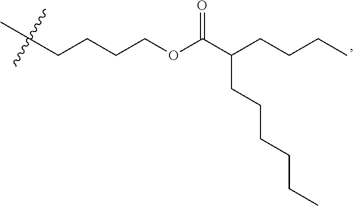 [SMILES: *CCCCOC(=O)C(CCCC)CCCCCC]

In some embodiments, R¹, R², R⁵, or R⁶ is

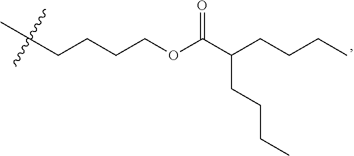 [SMILES: *CCCCOC(=O)C(CCCC)CCCC]

In some embodiments, R¹, R², R⁵, or R⁶ is

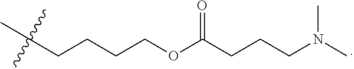 [SMILES: *CCCCOC(=O)CCCN(C)C]

In one embodiment, R³ and R⁴ are each independently H, C₁-C₆ alkyl, or C₁-C₆ alkyl alcohol. In some embodiments, R³ and R⁴ are each independently H, C₁-C₃ alkyl, or C₁-C₄ alkyl alcohol. In some embodiments, one of R³ or R⁴ is H and the other one of R³ or R⁴ is C₁-C₃ alkyl, or C₁-C₄ alkyl alcohol. In some embodiments, one of R³ or R⁴ is H and the other one of R³ or R⁴ is C₁-C₃ alkyl. In some embodiments, one of R³ or R⁴ is H and the other one of R³ or R⁴ is methyl. In some embodiments, one of R³ or R⁴ is H and the other one of R³ or R⁴ is C₁-C₄ alkyl alcohol. In some embodiments, one of R³ or R⁴ is H and the other one of R³ or R⁴ is C₂-C₄ alkyl alcohol. In some embodiments, one of R³ or R⁴ is H and the other one of R³ or R⁴ is ethyl alcohol. In some embodiments, one of R³ or R⁴ is H and the other one of R³ or R⁴ is n-propyl alcohol. In some embodiments, one of R³ or R⁴ is H and the other one of R³ or R⁴ is n-butyl alcohol.

In one embodiment, n is an integer ranging from 1 to 12. In some embodiments, n is an integer ranging from 1 to 10. In some embodiments, n is an integer ranging from 1 to 8. In some embodiments, n is an integer ranging from 1 to 6. In some embodiments, n is an integer of 1. In some embodiments, n is an integer of 2. In some embodiments, n is an integer of 3. In some embodiments, n is an integer of 4. In some embodiments, n is an integer of 5. In some embodiments, n is an integer of 6. In some embodiments, n is an integer ranging from 2 to 12. In some embodiments, n is an integer ranging from 2 to 11. In some embodiments, n is an integer ranging from 2 to 10. In some embodiments, n is an integer ranging from 2 to 9. In some embodiments, n is an integer ranging from 2 to 8. In some embodiments, n is an integer ranging from 2 to 7. In some embodiments, n is an integer ranging from 2 to 6. In some embodiments, n is an integer ranging from 3 to 6. In some embodiments, n is an integer ranging from 4 to 6. In some embodiments, n is an integer ranging from 5 to 6.

In one embodiment, x and w are each independently an integer ranging from 1 to 4. In some embodiments, x and w are an integer of 1. In some embodiments, x and w are an integer of 2. In some embodiments, x and w are an integer of 3. In some embodiments, x and w are an integer of 4. In some embodiments, x and w are each independently an integer ranging from 1 to 3. In some embodiments, x and w are each independently an integer ranging from 1 to 2.

In one embodiment, y and z are each independently an integer ranging from 1 to 6. In some embodiments, y and z are an integer of 1. In some embodiments, y and z are an integer of 2. In some embodiments, y and z are an integer of 3. In some embodiments, y and z are an integer of 4. In some embodiments, y and z are an integer of 5. In some embodiments, y and z are an integer of 6. In some embodiments, y and z are each independently an integer ranging from 1 to 5. In some embodiments, y and z are each independently an integer ranging from 1 to 4. In some embodiments, y and z are each independently an integer ranging from 1 to 3.

In one embodiment, the compound of formula (I)—(ID) has one of the following structures shown in Table A below.

TABLE A
List of Compounds of (I)-(ID)

| Entry | Structure |
|---|---|
| I-1 | 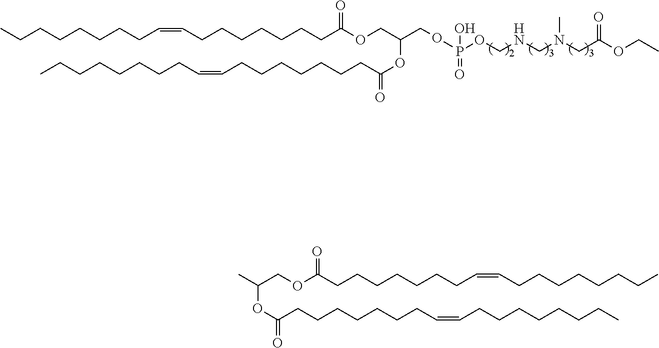 |
| I-2 | 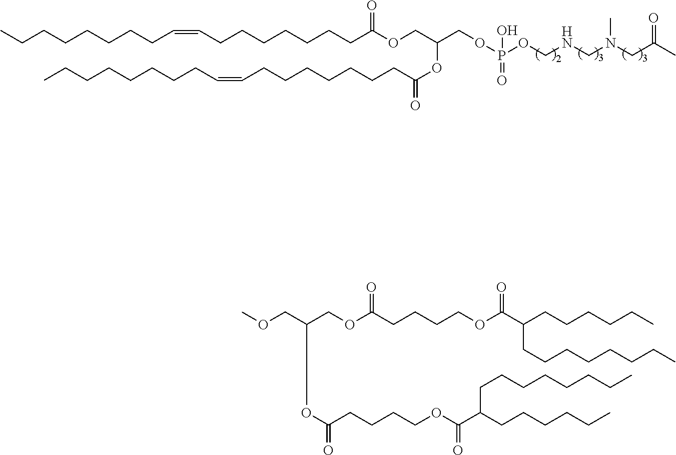 |
| I-3 | 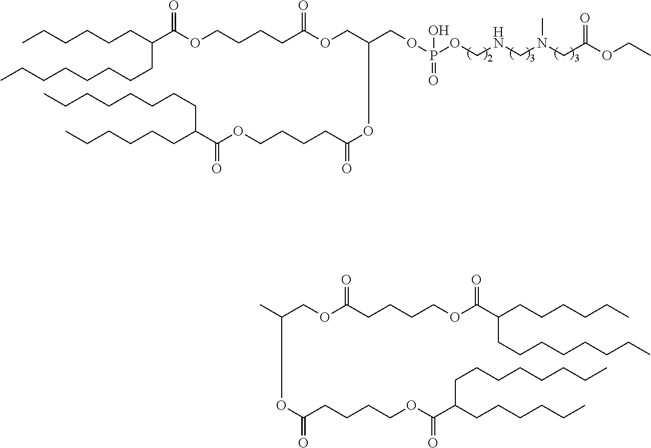 |
| I-4 | 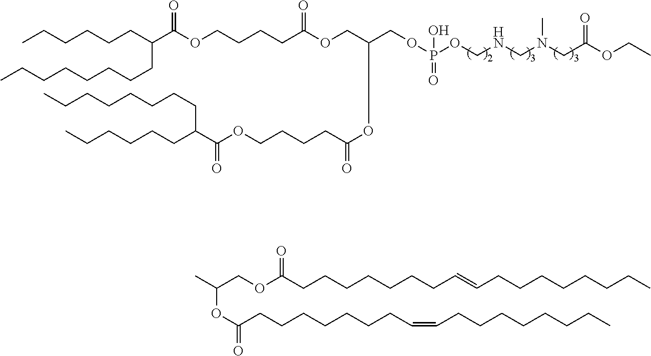 |

In one embodiment, the compound having

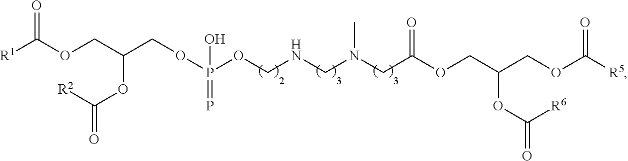

wherein R¹, R², R⁵, and R⁶ are

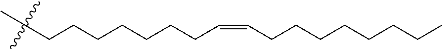 [SMILES: *CCCCCCC/C=C\CCCCCCCC]

corresponds to compound I-1 in Table A. In one embodiment, the compound having

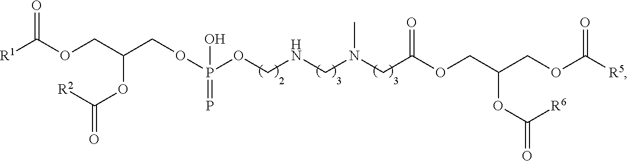

wherein R¹ and R² are

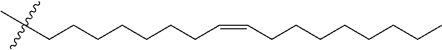 [SMILES: *CCCCCCC/C=C\CCCCCCCC]

and R⁵ and R⁶ are

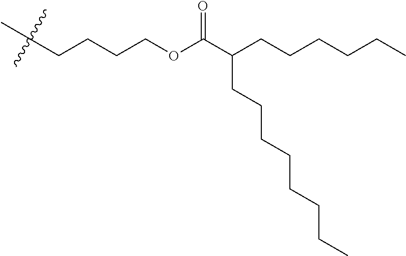 [SMILES: *CCCCOC(=O)C(CCCCCC)CCCCCCCC]

corresponds to compound I-2 in Table A. In one embodiment, the compound having

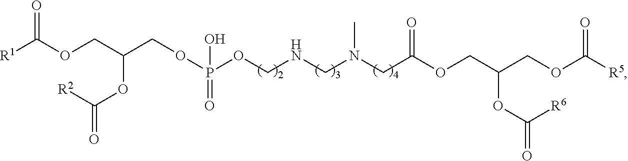

wherein R¹, R², R⁵, and R⁶ are

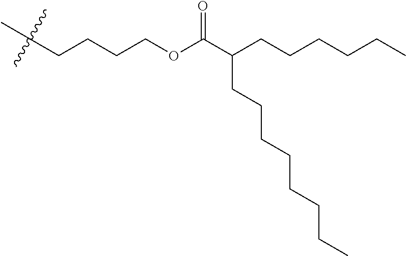 [SMILES: *CCCCOC(=O)C(CCCCCC)CCCCCCCC]

corresponds to compound I-3 in Table A. In one embodiment, the compound having

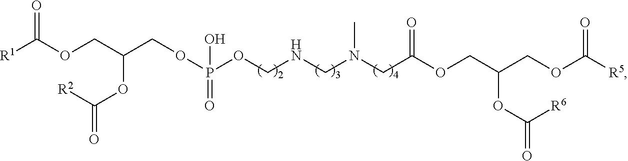

wherein R¹ and R² are

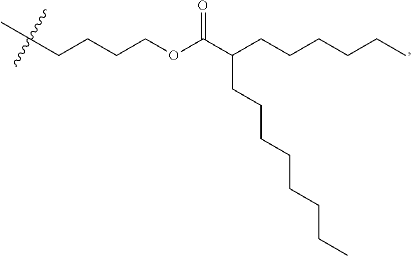 [SMILES: *CCCCOC(=O)C(CCCCCC)CCCCCCCC]

### R⁵ is

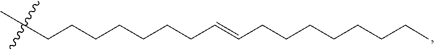 [SMILES: *CCCCCCC/C=C/CCCCCCCC]

### and R⁶ is

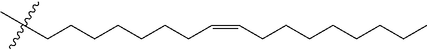 [SMILES: *CCCCCCC/C=C\CCCCCCCC]

corresponds to compound I-4 in Table A.

In one embodiment, the compounds shown in Table A are a pharmaceutically acceptable salt. In other embodiments, the pharmaceutically acceptable salt is a trifluoroacetic acid salt or hydrogen chloride salt. In other embodiments, the pharmaceutically acceptable salt is a trifluoroacetic acid salt. In other embodiments, the pharmaceutically acceptable salt is a hydrogen chloride salt.

In one embodiment, a compound having a structure of Formula (II) is provided:

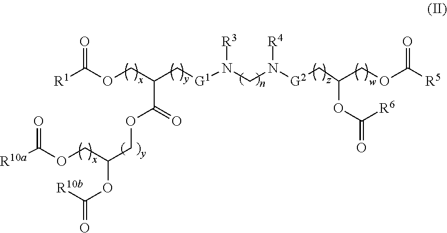

or stereoisomer, salt, or tautomer thereof, wherein G¹ is C₁-C₆ alkyl phosphate; G² is C₁-C₆ alkyl ester, C₁-C₆ alkyl amide, or C₁-C₆ alkyl phosphate; R¹, R⁵, R⁶, R^{10a}, and R^{10b} are each independently C₁₂-C₂₄ alkyl, C₁₂-C₂₄ alkylene, or C₁-C₆ alkyl, wherein the C₁-C₆ alkyl is substituted with —OC(═O)R⁷, —NR⁸C(═O)R⁷, or —C(═O)OR⁷; R³ and R⁴ are each independently H, C₁-C₆ alkyl, or C₁-C₆ alkyl alcohol; R⁷ is branched C₆-C₂₄ alkyl or C₂-C₁₆ heteroalkyl; R⁸ is H or C₁-C₆ alkyl; x and w are each independently an integer ranging from 1 to 4; n is an integer ranging from 1 to 12; and y, and z are each independently an integer ranging from 1 to 6.

In one embodiment, G¹ is C₁-C₆ alkyl phosphate; and G² is C₁-C₆ alkyl ester, C₁-C₆ alkyl amide, or C₁-C₆ alkyl phosphate. In some embodiments, G¹ is C₁-C₆ alkyl phosphate; and G² is C₁-C₆ alkyl ester. In some embodiments, G¹ is C₁-C₆ alkyl phosphate; and G² is C₁-C₆ alkyl amide. In some embodiments, G¹ is C₁-C₆ alkyl phosphate; and G² is C₁-C₆ alkyl phosphate.

In one embodiment, the compound has one of the following structures (IIA)-(IIB):

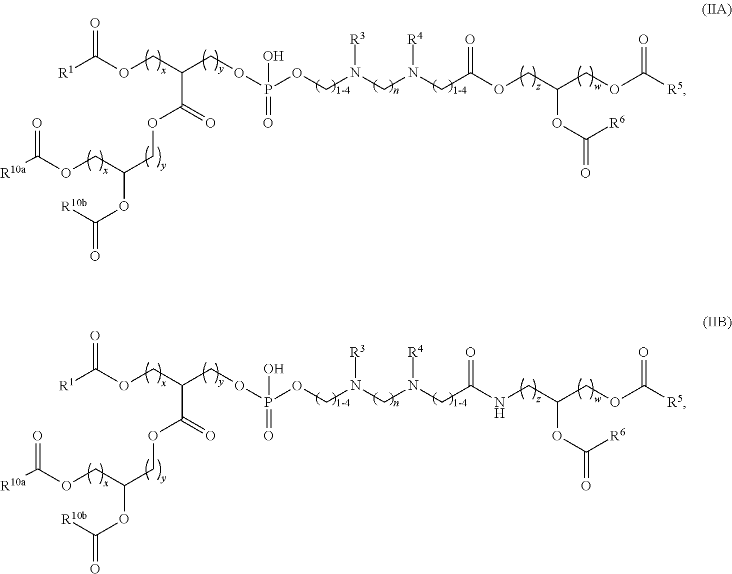

or a pharmaceutically acceptable salt or stereoisomer thereof.

In one embodiment, R¹, R⁵, R⁶, R^{10a}, and R^{10b} are each independently C₁₂-C₂₄ alkyl, C₁₂-C₂₄ alkylene, or C₁-C₆ alkyl, wherein the C₁-C₆ alkyl is substituted with —OC(═O)R⁷, —NR⁸C(═O)R⁷, or —C(═O)OR⁷. In some embodiments, R¹, R⁵, R⁶, R^{10a}, and R^{10b} are each independently C₁₂-C₂₄ alkylene or C₁-C₆ alkyl, wherein the C₁-C₆ alkyl is substituted with —OC(═O)R⁷, —NR⁸C(═O)R⁷, or —C(═O)OR⁷. In some embodiments, R¹, R⁵, R⁶, R^{10a}, and R^{10b} are each independently C₁₂-C₂₄ alkylene or C₁-C₆ alkyl, wherein the C₁-C₆ alkyl is substituted with —OC(═O)R⁷ or —NR⁸C(═O)R⁷. In some embodiments, R¹, R⁵, R⁶, R^{10a}, and R^{10b} are each independently C₁₂-C₂₄ alkylene or C₁-C₆ alkyl, wherein the C₁-C₆ alkyl is substituted with —OC(═O)R⁷.

In one embodiment, R⁷ has one of the following structures:

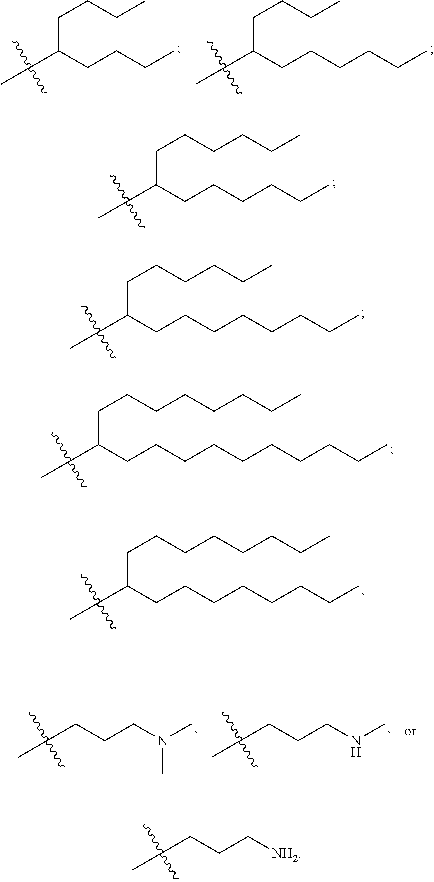

In some embodiments, R⁷ is

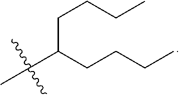 [SMILES: *C(CCCC)CCCC]

In some embodiments, R⁷ is

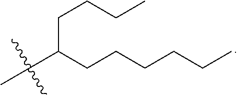 [SMILES: *C(CCCC)CCCCCC]

In some embodiments R⁷ is

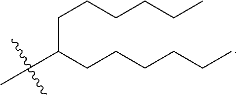 [SMILES: *C(CCCCCC)CCCCCC]

In some embodiments R⁷ is

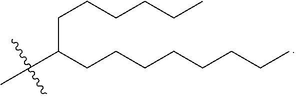 [SMILES: *C(CCCCCC)CCCCCCCC]

In some embodiments, R⁷ is

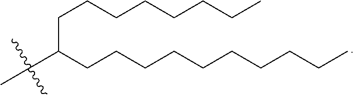 [SMILES: *C(CCCCCCCC)CCCCCCCCCC]

In some embodiments, R⁷ is

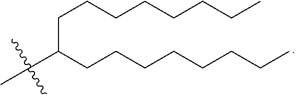 [SMILES: *C(CCCCCCCC)CCCCCCCC]

In some embodiments, R⁷ is

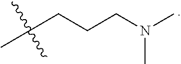 [SMILES: *CCCN(C)C]

In some embodiments, R⁷ is

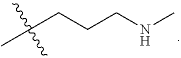 [SMILES: *CCCNC ; "."]

In some embodiments, R⁷ is

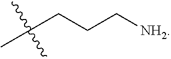 [SMILES: *CCCN]

In one embodiment, R⁷ has one of the following structures:

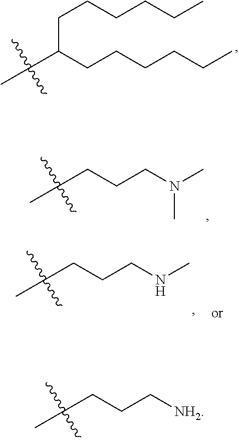

In one embodiment, R⁸ is H or C₁-C₃ alkyl. In some embodiments, R⁸ is H or methyl (—CH₃).

In one embodiment, R¹, R⁵, R⁶, R^{10a}, and R^{10b} each independently have one of the following structures:

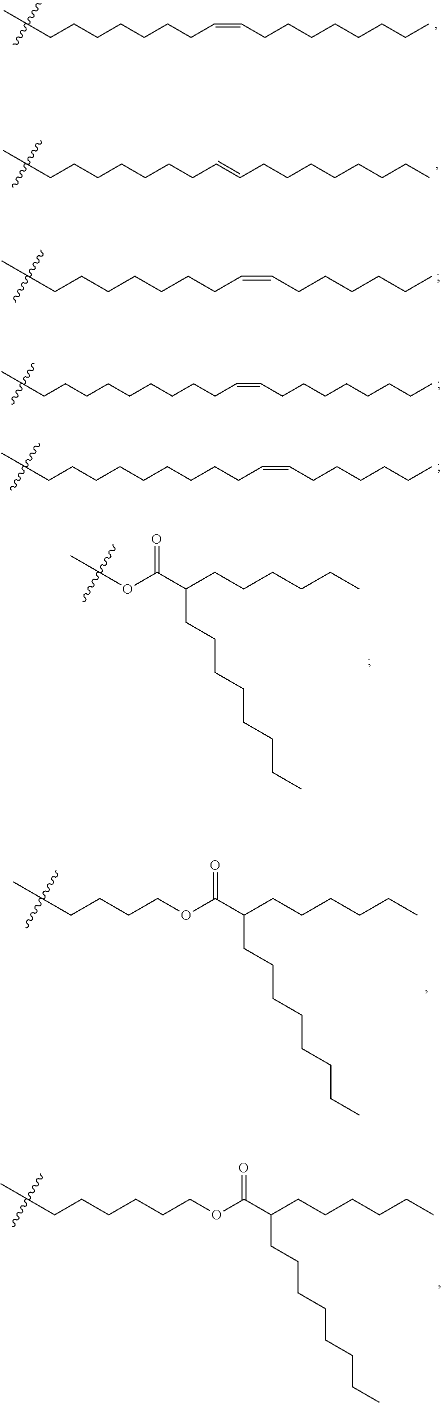

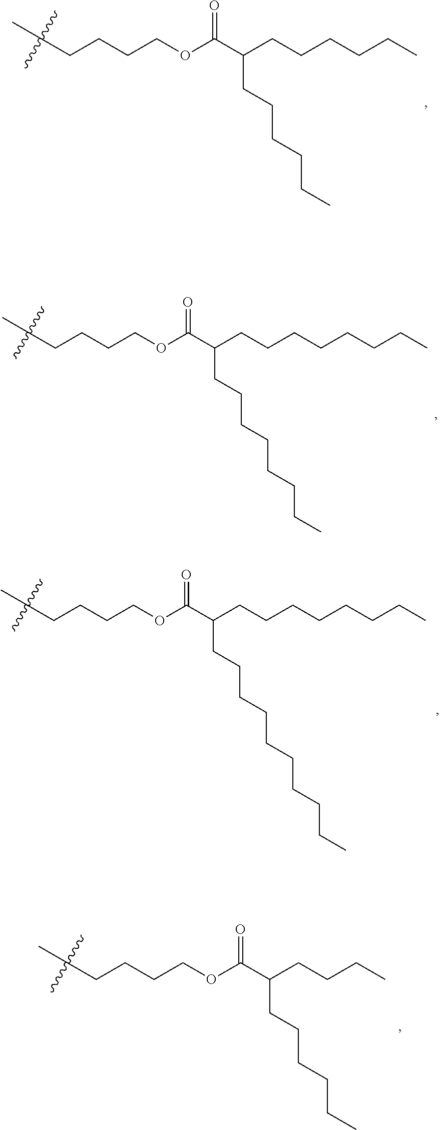 [SMILES: *CCCCOC(=O)C(CCCCCC)CCCCCC ; *CCCCOC(=O)C(CCCCCCCC)CCCCCCCC ; *CCCCOC(=O)C(CCCCCCCC)CCCCCCCCCC ; *CCCCOC(=O)C(CCCC)CCCCCC]

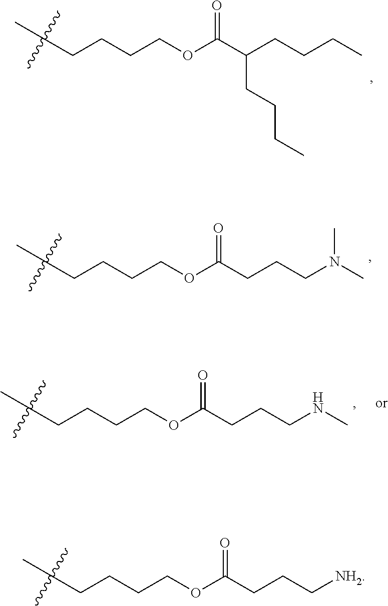 [SMILES: *CCCCOC(=O)C(CCCC)CCCC ; *CCCCOC(=O)CCCN(C)C ; *CCCCOC(=O)CCCNC ; *CCCCOC(=O)CCCN]

In some embodiments, R¹, R², R⁵, or R⁶ is

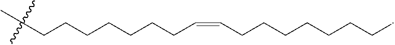

In some embodiments, R¹, R², R⁵, or R⁶ is

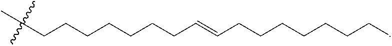

In some embodiments, R¹, R², R⁵, or R⁶ is

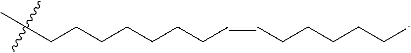

In some embodiments, R¹, R², R⁵, or R⁶ is

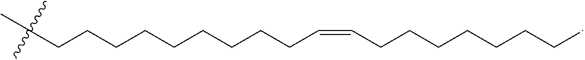

In some embodiments, R¹, R², R⁵, or R⁶ is

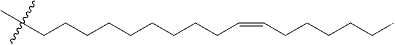

In some embodiments, R¹, R², R⁵, or R⁶ is

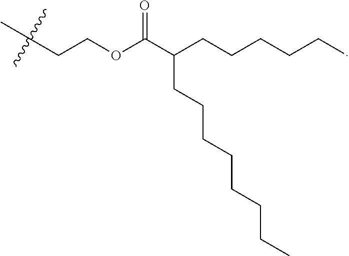

In some embodiments, R¹, R², R⁵, or R⁶ is

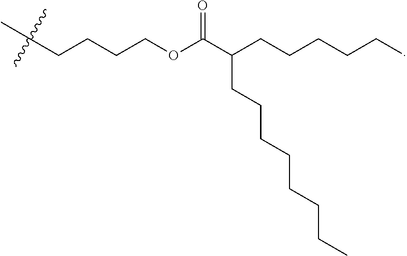

In some embodiments, R¹, R², R⁵, or R⁶ is

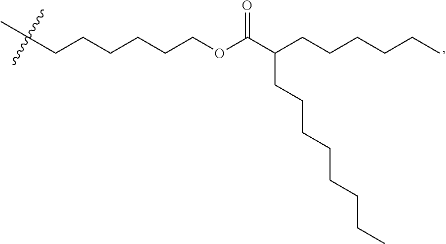 [SMILES: *CCCCCCOC(=O)C(CCCCCC)CCCCCCCC]

In some embodiments, R¹, R², R⁵, or R⁶ is

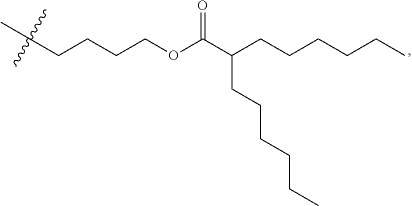 [SMILES: *CCCCOC(=O)C(CCCCCC)CCCCCC]

In some embodiments, R¹, R², R⁵, or R⁶ is

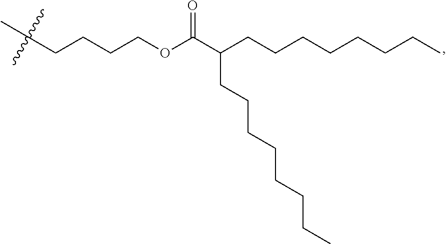 [SMILES: *CCCCOC(=O)C(CCCCCCCC)CCCCCCCC]

In some embodiments, R¹, R², R⁵, or R⁶ is

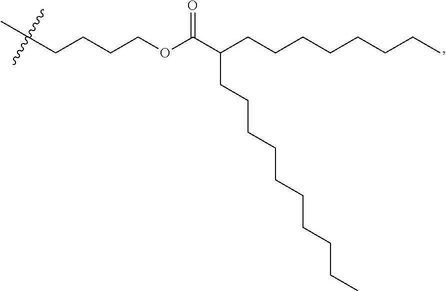 [SMILES: *CCCCOC(=O)C(CCCCCCCC)CCCCCCCCCC]

In some embodiments, R¹, R², R⁵, or R⁶ is

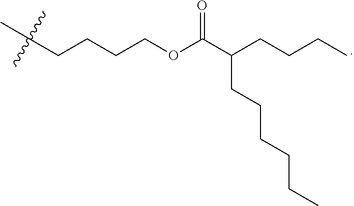 [SMILES: *CCCCOC(=O)C(CCCC)CCCCCC]

In some embodiments, R¹, R², R⁵, or R⁶ is

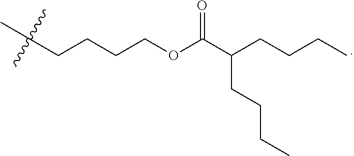 [SMILES: *CCCCOC(=O)C(CCCC)CCCC]

In some embodiments, R¹, R², R⁵, or R⁶ is

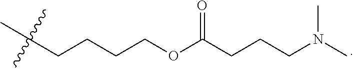 [SMILES: *CCCCOC(=O)CCCN(C)C]

In some embodiments, R¹, R², R⁵, or R⁶ is

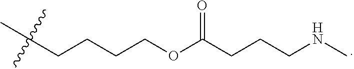 [SMILES: *CCCCOC(=O)CCCNC]

In some embodiments, R¹, R², R⁵, or R⁶ is

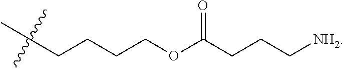 [SMILES: *CCCCOC(=O)CCCN]

In one embodiment, R³ and R⁴ are each independently H, C₁-C₆ alkyl, or C₁-C₆ alkyl alcohol. In some embodiments, R³ and R⁴ are each independently H, C₁-C₃ alkyl, or C₁-C₄ alkyl alcohol. In some embodiments, one of R³ or R⁴ is H and the other one of R³ or R⁴ is C₁-C₃ alkyl, or C₁-C₄ alkyl alcohol. In some embodiments, one of R³ or R⁴ is H and the other one of R³ or R⁴ is C₁-C₃ alkyl. In some embodiments, one of R³ or R⁴ is H and the other one of R³ or R⁴ is methyl. In some embodiments, one of R³ or R⁴ is H and the other one of R³ or R⁴ is C₁-C₄ alkyl alcohol. In some embodiments, one of R³ or R⁴ is H and the other one of R³ or R⁴ is C₂-C₄ alkyl alcohol. In some embodiments, one of R³ or R⁴ is H and the other one of R³ or R⁴ is ethyl alcohol. In some embodiments, one of R³ or R⁴ is H and the other one of R³ or R⁴ is n-propyl alcohol. In some embodiments, one of R³ or R⁴ is H and the other one of R³ or R⁴ is n-butyl alcohol.

In one embodiment, n is an integer ranging from 1 to 12. In some embodiments, n is an integer ranging from 1 to 6. In some embodiments, n is an integer of 1. In some embodiments, n is an integer of 2. In some embodiments, n is an integer of 3. In some embodiments, n is an integer of 4. In some embodiments, n is an integer of 5. In some embodiments, n is an integer of 6.

In one embodiment, x and w are each independently an integer ranging from 1 to 4. In some embodiments, x and w are an integer of 1. In some embodiments, x and w are an integer of 2. In some embodiments, x and w are an integer of 3. In some embodiments, x and w are an integer of 4.

In one embodiment, y and z are each independently an integer ranging from 1 to 6. In some embodiments, y and z are an integer of 1. In some embodiments, y and z are an integer of 2. In some embodiments, y and z are an integer of 3. In some embodiments, y and z are an integer of 4. In some embodiments, y and z are an integer of 5. In some embodiments, y and z are an integer of 6.

In one embodiment, the compound of formula (II)—(IIB) has one of the following structures shown in Table B below.

TABLE B
List of Compounds of (II)-(IIB)

| Entry | Structure |
|---|---|
| II-1 | 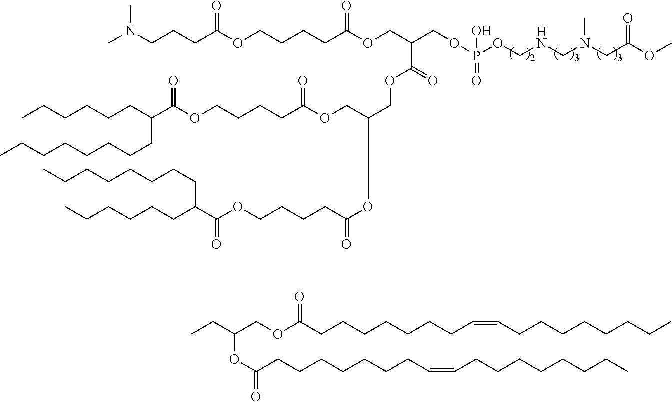 |
| II-2 | 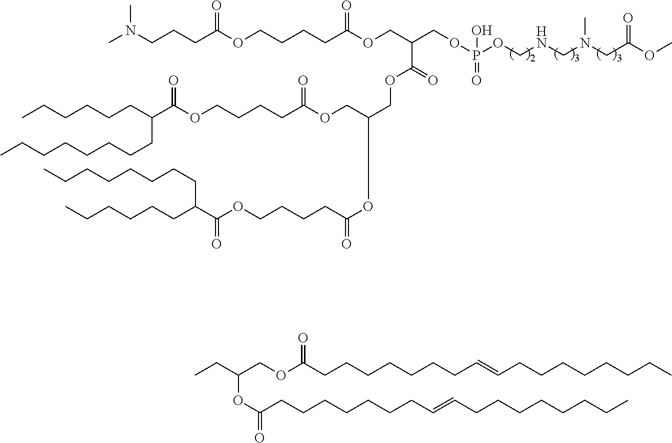 |
| II-3 | 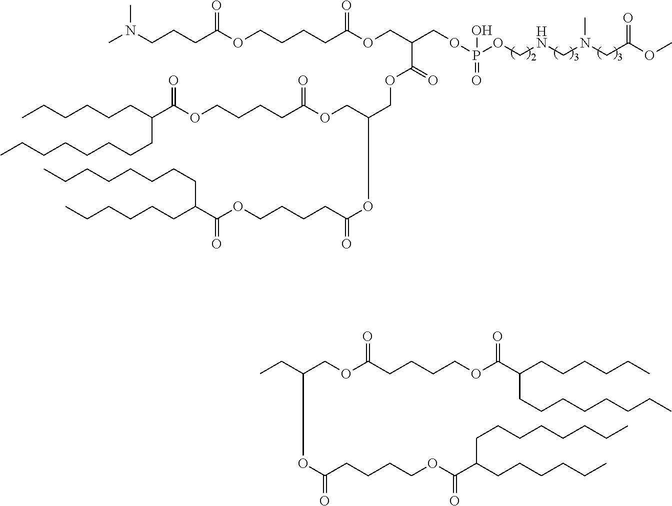 |
| II-4 | 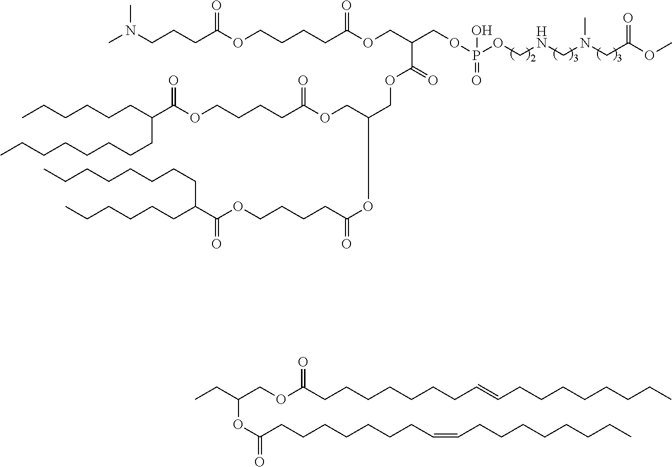 |
| II-5 | 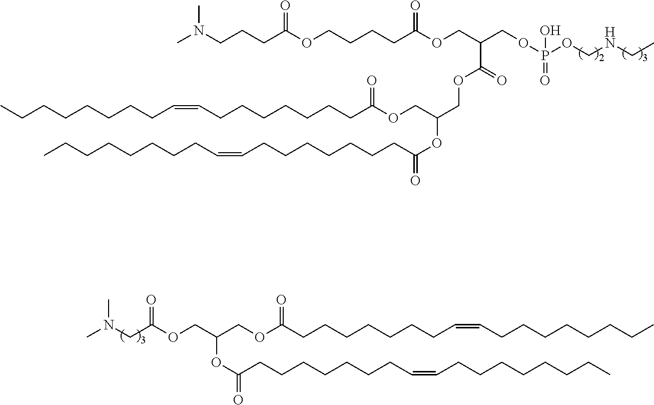 |
| II-6 | 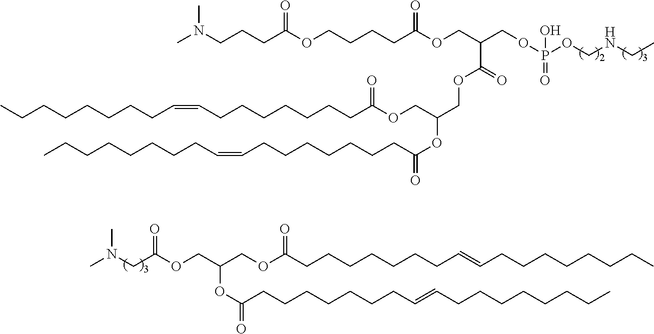 |
| II-7 | 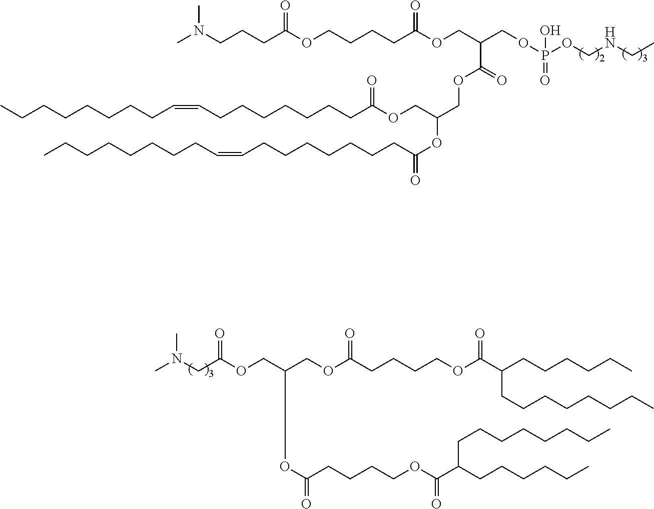 |

In one embodiment, the In one embodiment, the compound having

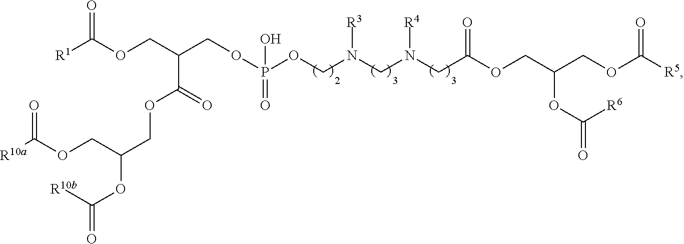

wherein R¹ is

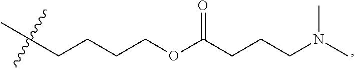 [SMILES: *CCCCOC(=O)CCCN(C)C]

R^{10a} and R^{10b} are

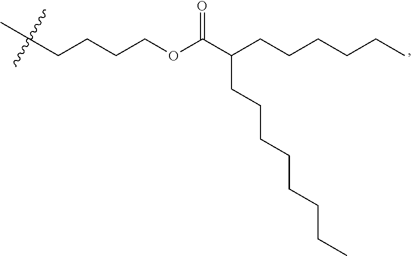 [SMILES: *CCCCOC(=O)C(CCCCCC)CCCCCCCC]

and R⁵ and R⁶ are

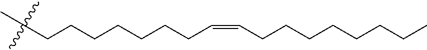 [SMILES: *CCCCCCC/C=C\CCCCCCCC]

corresponds to compound II-1 in Table B. In one embodiment, the compound having

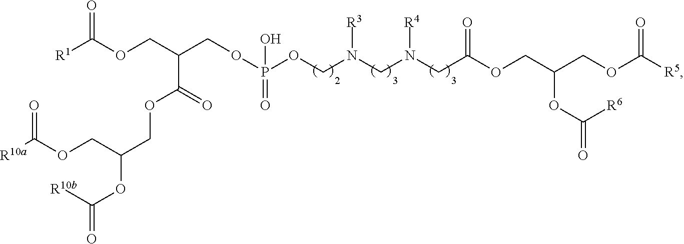

wherein R¹ is

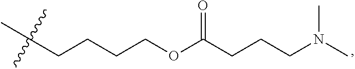 [SMILES: *CCCCOC(=O)CCCN(C)C]

R^{10a} and R^{10b} are

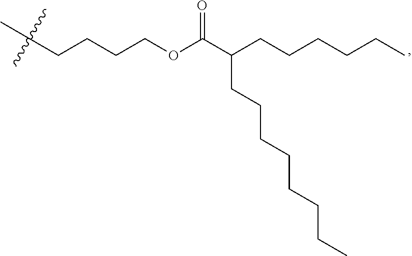 [SMILES: *CCCCOC(=O)C(CCCCCC)CCCCCCCC]

and R⁵ and R⁶ are

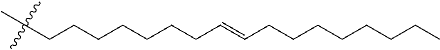 [SMILES: *CCCCCCC/C=C/CCCCCCCC]

corresponds to compound II-2 in Table B. In one embodiment, the compound having

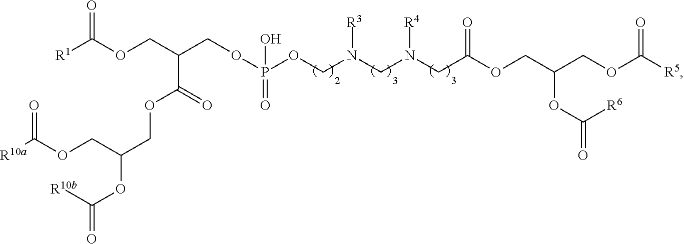

wherein R¹ is

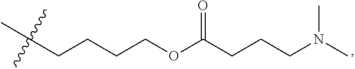 [SMILES: *CCCCOC(=O)CCCN(C)C]

R^{10a} and R^{10b} are

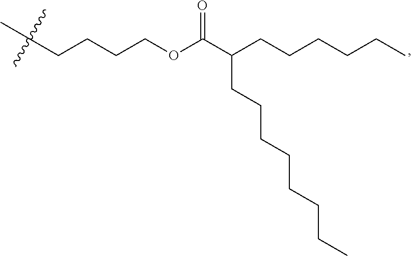 [SMILES: *CCCCOC(=O)C(CCCCCC)CCCCCCCC]

and R⁵ and R⁶ are

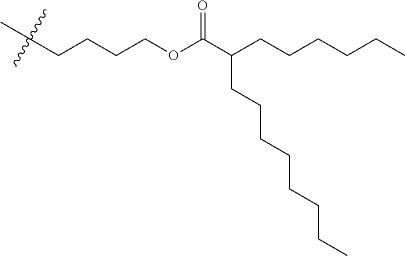 [SMILES: *CCCCOC(=O)C(CCCCCC)CCCCCCCC]

corresponds to compound II-3 in Table B. In one embodiment, the compound having

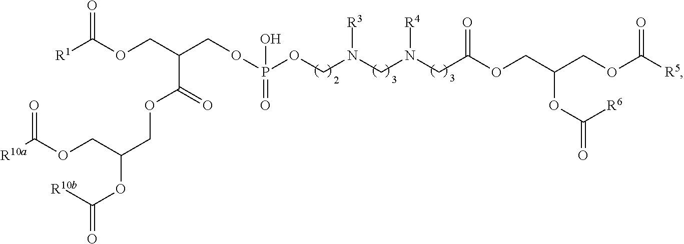

wherein R¹ is

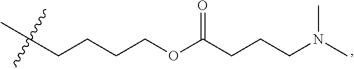 [SMILES: *CCCCOC(=O)CCCN(C)C]

R^{10a} and R^{10b} are

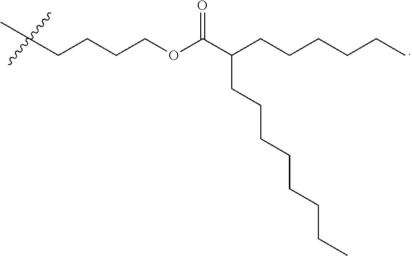 [SMILES: *CCCCOC(=O)C(CCCCCC)CCCCCCCC]

### R⁵ is

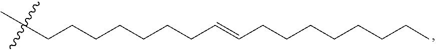 [SMILES: *CCCCCCC/C=C/CCCCCCCC]

### and R⁶ is

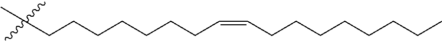 [SMILES: *CCCCCCC/C=C\CCCCCCCC]

corresponds to compound II-4 in Table B. In one embodiment, the compound having

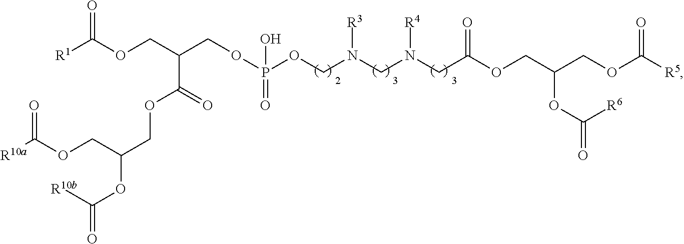

wherein R¹ is

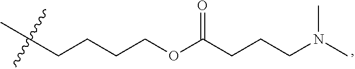 [SMILES: *CCCCOC(=O)CCCN(C)C]

R^{10a} and R^{10b} are

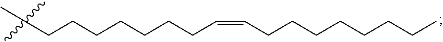 [SMILES: *CCCCCCC/C=C\CCCCCCCC]

and R⁵ and R⁶ are

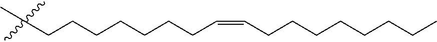 [SMILES: *CCCCCCC/C=C\CCCCCCCC]

corresponds to compound II-5 in Table B. In one embodiment, the compound having

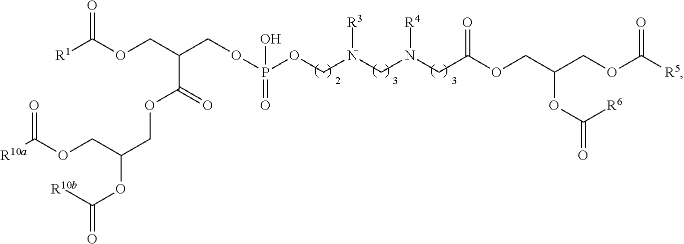

wherein R¹ is

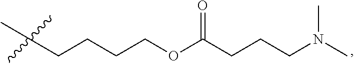 [SMILES: *CCCCOC(=O)CCCN(C)C]

R^{10a} and R^{10b} are

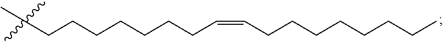 [SMILES: *CCCCCCC/C=C\CCCCCCCC]

and R⁵ and R⁶ are

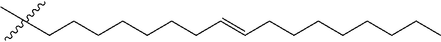 [SMILES: *CCCCCCC/C=C/CCCCCCCC]

corresponds to compound II-6 in Table B. In one embodiment, the compound having

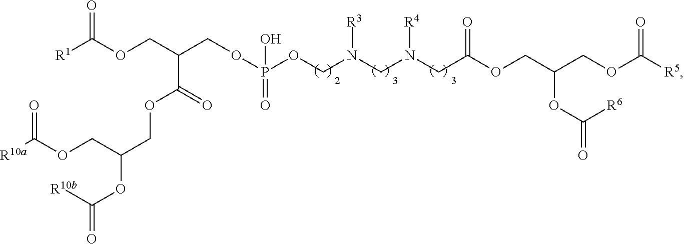

wherein R¹ is

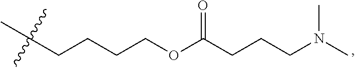 [SMILES: *CCCCOC(=O)CCCN(C)C]

R^{10a} and R^{10b} are

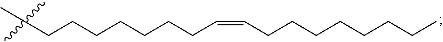 [SMILES: *CCCCCCC/C=C\CCCCCCCC]

and R⁵ and R⁶ are

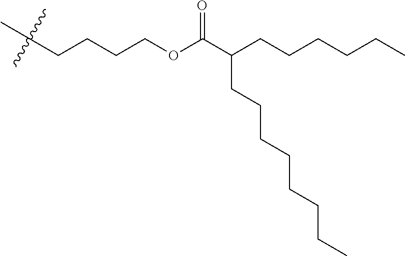 [SMILES: *CCCCOC(=O)C(CCCCCC)CCCCCCCC]

corresponds to compound II-7 in Table B.

As can be appreciated, the ionizable lipids described herein enable the development of new therapies for disease without the need for exotic chemistry or specialized reagents or manufacturing techniques.

### III Compositions and Uses

In some aspects, the dual lipids and methods of using the same, are included in compositions, are included in compositions, such as lipid-nanoparticle (LNP) compositions, for delivery of molecules, particularly therapeutic molecules including therapeutic nucleic acids. In embodiments, a composition comprising a dual lipid as described herein is used in a composition, such as a lipid-nanoparticle (LNP) composition, for delivery of a nucleic acid to a target cell and/or tissue, such as by transfection of the target cells and/or tissues. More particularly, LNP compositions comprising a dual lipid described herein and an RNA are administered to a subject for treatment of a condition or disorder. Accordingly, in aspects, the current technology is related to LNP compositions comprising the described dual lipids and methods of using such compositions for delivery of nucleic acids to cells and/or tissues, such as by transfecting cells and/or tissues with LNP, dual lipid compositions of the present technology.

In embodiments, lipid-based delivery systems and methods including, but not limited to, lipid nanoparticles (LNP) comprising the dual lipids of the present technology, may provide an approach to stabilize and deliver nucleic acids. In embodiments, design features, such as optimal particle size, encapsulation efficiencies, robust manufacturing process, different lipophilicity and neutral surface charge, can be further advanced to provide efficient lipid-based delivery systems of nucleic acids with the dual lipids of the present technology.

In some embodiments, lipid-based delivery systems and methods, such as, but not limited to, lipid nanoparticle (LNP) compositions and methods of using the same, may be used in conjunction with the dual lipids described herein. Such compositions comprising a dual lipid, such as an LNP composition, and methods using the same provide an approach to stabilize and/or deliver nucleic acids, and other therapeutic molecules. In embodiments, dual lipid compositions and LNP compositions comprising a dual lipid, and methods using the same, include design features, such as optimal particle size, high encapsulation efficiency, robust manufacturing process, and optimal lipophilicity and surface charge, to provide efficient lipid-based delivery systems for nucleic acids and other therapeutic molecules. In some embodiments, dual lipid compositions and LNP compositions comprising a dual lipid, and methods of use, include additional ingredients and components. In some embodiments, the additional ingredients are present inside the lipid nanoparticle itself.

In some embodiments, the dual lipid compositions and/or LNP compositions comprising a dual lipid, and methods of use provided herein, provide delivery of a nucleic acid to a target cell or tissue, wherein the nucleic acid may be selected from a small interfering RNA (siRNA), an asymmetrical interfering RNA (aiRNA), a microRNA (miRNA), a Dicersubstrate RNA (dsRNA), a small hairpin RNA (shRNA), a messenger RNA (mRNA), a guide RNA (gRNA), a plasmid DNA (pDNA), an antisense oligodeoxynucleotide (ODN), RNA or DNA vaccine, and mixtures thereof.

Other embodiments are directed to pharmaceutical compositions. In an embodiment, the pharmaceutical composition comprises any one (or more) of the foregoing compounds and a pharmaceutically acceptable carrier. In some embodiments, the pharmaceutical composition is formulated for oral administration. In other embodiments, the pharmaceutical composition is formulated for injection. In still more embodiments, the pharmaceutical compositions comprise a compound as disclosed herein and an additional therapeutic agent (e.g., anticancer agent). Non-limiting examples of such additional therapeutic agents are described herein below.

Suitable routes of administration include, but are not limited to, oral, intravenous, rectal, aerosol, parenteral, ophthalmic, pulmonary, transmucosal, transdermal, vaginal, optical, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the compound is delivered in a targeted drug delivery system, for example, in a liposome coated with and organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

In treatment methods according to embodiments of the disclosure, an effective amount of at least one compound of Formula (I)—(IIB) is administered to a subject suffering from or diagnosed as having such a disease, disorder, or medical condition. Effective amounts or doses may be ascertained by methods such as modeling, dose escalation studies or clinical trials, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician.

The compounds according to the disclosure are effective over a wide dosage range. For example, in the treatment of adult humans, dosages from about 0.001 to 0.1 mg, 0.01 to 0.1 mg, 0.5 to 5 mg, 0.5 to 10 mg, 0.01-10 mg, 0.1 to 10 mg, 10 to 5000 mg, 100 to 5000 mg, 1000 mg to 4000 mg per day, or 1000 to 3000 mg per day are examples of dosages that are used in some embodiments. The exact dosage will depend upon the route of administration, the form in which the compound is administered, the subject to be treated, the body weight of the subject to be treated, and the preference and experience of the attending physician.

In some embodiments, compounds of the disclosure are administered in a single dose. In an embodiment, the single dose is administered orally. In another embodiment, the single dose is administered topically. However, other routes are used as appropriate. In some embodiments, compounds of the disclosure are administered in multiple doses. In some embodiments, dosing is about once, twice, three times, four times, five times, six times, or more than six times per day. In other embodiments, dosing is about once a month, once every two weeks, once a week, or once every other day. In another embodiment compounds of the disclosure and another agent (e.g., anti-cancer agent) are administered together about once per day to about 6 times per day. In another embodiment the administration of compounds of the disclosure and an agent continues for less than about 7 days. In yet another embodiment the administration continues for more than about 6, 10, 14, 28 days, two months, six months, or one year. In some cases, continuous dosing is achieved and maintained as long as necessary.

Administration of compounds of the disclosure may continue as long as necessary. In some embodiments, compounds of the disclosure are administered for more than 1, 2, 3, 4, 5, 6, 7, 14, or 28 days. In some embodiments, compounds of the disclosure are administered for less than 28, 14, 7, 6, 5, 4, 3, 2, or 1 day. In some embodiments, compounds of the disclosure are administered chronically on an ongoing basis, e.g., for the treatment of chronic effects.

In some embodiments, the compounds of the disclosure are administered in individual dosage forms. It is known in the art that due to intersubject variability in compound pharmacokinetics, individualization of dosing regimen is necessary for optimal therapy.

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the disclosed compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising one or more compounds of Formula (I)—(IIB), and a pharmaceutically acceptable carrier. Also provided herein are pharmaceutical compositions comprising one or more compounds selected from compounds of Formula (I)—(IIB) and pharmaceutically acceptable diluent(s), excipient(s), and carrier(s). In certain embodiments, the compounds described are administered as pharmaceutical compositions in which one or more compounds selected from compounds of Formula (I)—(IIB) are mixed with other active ingredients, as in combination therapy. Encompassed herein are all combinations of actives set forth in the combination therapies section below and throughout this disclosure. In specific embodiments, the pharmaceutical compositions include one or more compounds of Formula (I)—(IIB).

A pharmaceutical composition, as used herein, refers to a mixture of one or more compounds selected from compounds of Formula (I)—(IIB) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, therapeutically effective amounts of one or more compounds selected from compounds of Formula (I)—(IIB) provided herein are administered in a pharmaceutical composition to a mammal having a disease, disorder or medical condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, one or more compounds selected from compounds of Formula (I)—(IF) are formulated in aqueous solutions. In specific embodiments, the aqueous solution is selected from, by way of example only, a physiologically compatible buffer, such as Hank's solution, Ringer's solution, or physiological saline buffer. In other embodiments, one or more compounds selected from compounds of Formula (I)—(IIB) are formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or non-aqueous solutions. In specific embodiments, such solutions include physiologically compatible buffers and/or excipients.

In another embodiment, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross linked croscarmellose sodium, polyvinylpyrrolidone, agar, or alginic acid or a salt thereof such as sodium alginate.

In one embodiment, the oral dosage forms, such as a pill, capsule or tablet, comprises one or more suitable layers or coatings. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push fit capsules made of gelatin, as well as soft, sealed capsules made of gelatin and a plasticizer, such as glycerol or sorbitol. In specific embodiments, push fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In still other embodiments, the compounds described herein are formulated for parental injection, including formulations suitable for bolus injection or continuous infusion. In specific embodiments, formulations for injection are presented in unit dosage form (e.g., in ampoules) or in multi dose containers. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water soluble form. In additional embodiments, suspensions of one or more compounds selected from compounds of Formula (I)—(IIB) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

Pharmaceutical compositions include at least one pharmaceutically acceptable carrier, diluent or excipient, and one or more compounds selected from compounds of Formula (I)—(IIB) as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compound(s) with one or more inert, pharmaceutically acceptable excipients or carriers to form a solid, semi-solid or liquid composition. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, ointments, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical compositions comprising one or more compounds selected from compounds of Formula (I)—(IIB) illustratively takes the form of a liquid where the agents are present in solution, in suspension or both. Typically when the composition is administered as a suspension, a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, aqueous suspensions contain one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally, include solubilizing agents to aid in the solubility of one or more compounds selected from compounds of Formula (I)—(IIB). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Compositions may include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Compositions may include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained release system, such as semipermeable matrices of solid hydrophobic polymers containing the therapeutic agent. Various sustained release materials are useful herein. In some embodiments, sustained release capsules release the compounds for a few weeks up to over 100 days. Depending on the chemical nature and the biological stability of the therapeutic reagent, additional strategies for protein stabilization are employed.

In certain embodiments, the compositions and/or formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

In some embodiments, the concentration of one or more compounds selected from compounds of Formula (I)—(IIB) provided in the pharmaceutical compositions is greater than 95%, 90%, 85%, 80%, 75%, 70%, 65%, 60%, 55%, 50%, 45%, 40%, 35%, 30%, 25%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25%, 7%, 6.75%, 6.50%, 6.25% 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, v/v, or mole/mole (mole fraction). In another embodiment, the amount of a compound selected from compounds of Formula (I)—(IIB) in the pharmaceutical compositions is an amount between about any two of the values recited in the preceding sentence, for example, between about 2-70 w/w, w/v, v/v, or mole %, 2-80 w/w, w/v, v/v, or mole/mole, 2-85 w/w, w/v, v/v, or mole %, 3.5-80 w/w, w/v, v/v, or mole/mole, 1-30 w/w, w/v, v/v, or mole/mole, 1-50 w/w, w/v, v/v, or mole/mole, 1-60 w/w, w/v, v/v, or mole/mole, 1-70 w/w, w/v, v/v, or mole/mole, 1-80 w/w, w/v, v/v, or mole/mole, 1-90 w/w, w/v, v/v, or mole/mole, etc.

In some embodiments, the concentration of one or more compounds selected from compounds of Formula (I)—(IIB) provided in the pharmaceutical compositions of the present disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v, v/v, or mole/mole.

In some embodiments, the amount the one or more compounds selected from compounds of Formula (I)—(IIB) provided in the pharmaceutical compositions of the present disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the one or more compounds selected from compounds of Formula (I)—(IIB) provided in the pharmaceutical compositions of the present disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g.

Packaging materials for use in packaging pharmaceutical compositions described herein include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical compositions are presented in a pack or dispenser device which contains one or more unit dosage forms containing a compound provided herein. The pack for example contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound provided herein formulated in a compatible pharmaceutical carrier are prepared, placed in an appropriate container, and labeled for treatment of an indicated condition.

As mentioned above, the compounds and compositions of the disclosure will find utility in a broad range of diseases and conditions mediated by protein kinases, including diseases and conditions mediated by kinase. Such diseases may include by way of example and not limitation, cancers such as lung cancer, NSCLC (non small cell lung cancer), oat-cell cancer, bone cancer, pancreatic cancer, skin cancer, dermatofibrosarcoma protuberans, cancer of the head and neck, cutaneous or intraocular melanoma, uterine cancer, ovarian cancer, colo-rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's Disease, hepatocellular cancer, cancer of the esophagus, cancer of the small intestine, cancer of the endocrine system (e.g., cancer of the thyroid, pancreas, parathyroid or adrenal glands), sarcomas of soft tissues, cancer of the urethra, cancer of the penis, prostate cancer (particularly hormone-refractory), chronic or acute leukemia, solid tumors of childhood, hypereosinophilia, lymphocytic lymphomas, cancer of the bladder, cancer of the kidney or ureter (e.g., renal cell carcinoma, carcinoma of the renal pelvis), pediatric malignancy, neoplasms of the central nervous system (e.g., primary CNS lymphoma, spinal axis tumors, medulloblastoma, brain stem gliomas or pituitary adenomas), Barrett's esophagus (pre-malignant syndrome), neoplastic cutaneous disease, psoriasis, mycoses fungoides, and benign prostatic hypertrophy, diabetes related diseases such as diabetic retinopathy, retinal ischemia, and retinal neovascularization, hepatic cirrhosis, angiogenesis, cardiovascular disease such as atherosclerosis, immunological disease such as autoimmune disease and renal disease.

In some embodiments, a pharmaceutical composition has a compound described above and a pharmaceutically acceptable carrier including, for example, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In some embodiments, a method treating a disease or disorder, the method includes administering an effective amount of the compound or the pharmaceutical composition described herein to a subject in need thereof. In some embodiments, the disease or disorder is a kinase-expressing cancer. In some specific embodiments, the cancer is bladder cancer. In some other specific embodiments, the cancer is prostate cancer. In some other specific embodiments, the cancer is a hematological malignancy such as acute myeloid leukemia. In some other specific embodiments, the disease or disorder is an autoimmune or inflammatory disease.

In one embodiment, a pharmaceutical composition comprises the compound of formula (I)—(IIB) and a therapeutic agent comprising a nucleic acid.

In one embodiment, the pharmaceutical composition comprising the compound of formula (I)—(IIB) and a therapeutic agent further comprises a helper lipid, a stabilization lipid, or a structural lipid.

In one embodiment, the helper lipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and mixtures thereof.

In one embodiment, the stabilization lipid is 1-(monomethoxy-polyethyleneglycol)-2,3-dimyristoylglycerol (PEG-DMG), with an average PEG molecular weight of 2000.

In one embodiment, the structural lipid is selected from the group consisting of cholesterol, cholesterol derivatives, fecosterol, sitosterol, ergosterol, campesterol, stigmasterol, brassicasterol, tomatidine, ursolic acid, alpha tocopherol, and mixtures thereof.

In one embodiment, the nucleic acid is selected from small interfering RNA (siRNA), an asymmetrical interfering RNA (aiRNA), a microRNA (miRNA), a Dicer-substrate RNA (dsRNA), a small hairpin RNA (shRNA), or a messenger RNA (mRNA).

In one embodiment, the nucleic acid is mRNA.

In one embodiment, a pharmaceutical composition comprising a compound of formula (I)—(IIB) and a second therapeutic agent.

In one embodiment, a method of administering a therapeutic agent comprising a nucleic acid to a patient in need thereof, the method comprising administering the pharmaceutical composition to the patient.

In one embodiment, the patient is animal. In other embodiments, the patient is human.

In one embodiment, a lipid nanoparticle comprising the compound of formula (I)—(IIB) and a therapeutic agent comprising a nucleic acid.

In one embodiment, the therapeutic agent of the lipid nanoparticle is mRNA.

The lipid component of a lipid-nanoparticle composition may include one or more structural lipids. Structural lipids can be selected from the group consisting of, but not limited to cholesterol, fecosterol, sitosterol, ergosterol, campesterol, stigmasterol, brassicasterol, tomatidine, ursolic acid, alpha-tocopherol, and mixtures thereof. In some embodiments, the structural lipid is cholesterol. In some embodiments, the structural lipid includes cholesterol and a corticosteroid (such as prednisolone, dexamethasone, prednisone, and hydrocortisone), or a combination thereof.

The structural lipids can also include natural and synthetic cholesterol derivatives. Some examples of natural cholesterol derivatives include, but not limited to, (70-OHC, 22 (R)-hydroxycholesterol (22R—OHC), 24 (S)-hydroxycholesterol (24 (S)—OHC)). Synthetic cholesterol derivates may include, but not limited to, (22(R)-hydroxy-Δ⁹-cholestanol (22R—ISO—OHC), ((23-(4-Methylfuran-2,5-dione)-3α-hydroxy-24-nor-5β-cholane (LITHO 1a), 23-(4-Methylfuran-2,5-dione)-3α,7α-dihydroxy-24-nor-5β-cholane (CHENO 1b), 23-(4-Methyl-1H-pyrrole-2,5-dione)-3α-hydroxy-24-nor-5β-cholane (LITOMAL 7a), 23-(4-Methyl-1H-pyrrole-2,5-dione)-3α,7α, 12α-trihydroxy-24-nor-5β-cholane (COLMAL 7f) and ethanol maleimide derivatives of litocholic and chenodeoxycholic acid (LITOMET, CHENOMET)) (146,147). The systematic name of LITOMET is (23-((2-hydroxyethyl)-4-methyl-1H-pyrrole-2,5-dione)-3α-hydroxy-24-nor-5β-cholane) and the systematic name of CHENOMET is (23-((2-hydroxyethyl)-4-methyl-1H-pyrrole-2,5-dione)-3α,7α-dihydroxy-24-nor-5β-cholane).

The lipid-nanoparticle composition of the disclosure, include nucleic acids, such as, but not limited to, small interfering RNA (siRNA), an asymmetrical interfering RNA (aiRNA), a microRNA (miRNA), miRNA inhibitors (antagomirs/antimirs), a dicer-substrate RNA (dsRNA), a small hairpin RNA (shRNA), a messenger RNA (mRNA), multivalent RNA, and mixtures thereof.

The lipid-nanoparticle composition of the disclosure may be prepared by mixing processes such as, but not limited to, microfluidics and T-junction mixing of two fluid streams, one of which contains the nucleic acid and the other has the lipid components. Such mixing process induces nano-precipitation and particle formation.

The lipid-nanoparticle composition of the disclosure can be characterized using a Zetasizer to determine the particle size, the polydispersity index (PDI) and zeta potential. In some embodiments, the zeta potential of the lipid-nanoparticle composition may be from about −10 mV to about +20 mV, from about −10 mV about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 m V to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

Ultraviolet-visible spectroscopy can be used to determine the concentration of the nucleic acid in the nanoparticle compositions.

The lipid-nanoparticle composition of the disclosure can induce expression of a desired protein both in vitro and in vivo by contacting cells with a lipid nanoparticle comprising one or more of the ionizable lipids described herein, wherein the lipid nanoparticle is encapsulated or is associated with a nucleic acid that is expressed to produce the desired protein (e.g., mRNA).

The lipid-nanoparticle composition of the disclosure can decrease the expression of target genes and proteins both in vitro and in vivo by contacting cells with a lipid nanoparticle comprising one or more of the ionizable lipids described herein, wherein the lipid nanoparticle is encapsulated or is associated with a nucleic acid that reduces target gene expression (e.g., siRNA).

The lipid-nanoparticle composition of the disclosure can down-regulate or silence the expression of target genes and proteins both in vitro and in vivo by contacting cells with a lipid nanoparticle comprising one or more of the ionizable lipids described herein, wherein the lipid nanoparticle is encapsulated or is associated with a nucleic acid that down-regulate or silence target gene expression.

The lipid-nanoparticle composition, in some embodiments, may comprise a neutral a structural lipid, such as cholesterol, fecosterol, sitosterol, campesterol, stigmasterol, brassicasterol, ergosterol, tomatidine, tomatine, ursolic acid and/or alpha-tocopherol. The lipid-nanoparticle composition can exclude helper lipids, phospholipids, non-ionic lipids, non-cationic lipids, cholesterol or derivatives of cholesterol, or PEG-lipids, or any combination thereof.

In one aspect, the lipids, such as transfection lipids, described herein relate to a lipid-nanoparticle composition or lipid nanoparticle comprising an ionizable lipid of any one of Formula (I) to Formula (IIB). The lipid-nanoparticle composition or lipid nanoparticle can further comprise a helper lipid, a stabilization lipid, a structural lipid, and/or a nucleic acid. The lipid-nanoparticle composition or lipid nanoparticle can, in embodiments, exclude one or more of helper lipids, phospholipids, non-ionic lipids, non-cationic lipids, cholesterol and/or a derivative of cholesterol, and/or PEG-lipids, or any combination thereof.

In some embodiments, the lipid-nanoparticle composition or lipid nanoparticle excludes helper lipids, phospholipids, non-ionic lipids, non-cationic lipids, PEG-lipids, and/or cholesterol or derivatives of cholesterol, or any combination thereof. In some embodiments, the lipid-nanoparticle composition or lipid nanoparticle excludes non-ionic lipids. In some embodiments, the lipid-nanoparticle composition or lipid nanoparticle excludes non-cationic lipids. In some embodiments, the lipid-nanoparticle composition or lipid nanoparticle excludes phospholipids. In some embodiments, the lipid-nanoparticle composition or lipid nanoparticle excludes cholesterol or derivatives of cholesterol. In some embodiments, the lipid-nanoparticle composition or lipid nanoparticle excludes phospholipids and cholesterol or derivatives of cholesterol. In some embodiments, the lipid-nanoparticle composition or lipid nanoparticle excludes PEG-lipids. In some embodiments, the lipid-nanoparticle composition or lipid nanoparticle excludes PEG-lipids, phospholipids, and cholesterol or derivatives of cholesterol.

In some embodiments, the lipid-nanoparticle composition excludes helper lipids, phospholipids, non-ionic lipids, non-cationic lipids, PEG-lipids, and/or cholesterol or derivatives of cholesterol, or any combination thereof. In some embodiments, the lipid-nanoparticle composition excludes non-ionic lipids. In some embodiments, the lipid-nanoparticle composition excludes non-cationic lipids. In some embodiments, the lipid-nanoparticle composition excludes phospholipids. In some embodiments, the lipid-nanoparticle composition excludes cholesterol or derivatives of cholesterol. In some embodiments, the lipid-nanoparticle composition excludes phospholipids and cholesterol or derivatives of cholesterol. In some embodiments, the lipid-nanoparticle composition excludes PEG-lipids. In some embodiments, the lipid-nanoparticle composition excludes PEG-lipids, phospholipids, and cholesterol or derivatives of cholesterol.

### IV. Methods of Preparation

Preparation methods for the above compounds and compositions are described herein below and/or known in the art.

It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds may not possess pharmacological activity as such, they may be administered to a mammal and thereafter metabolized in the body to form compounds which are pharmacologically active. Such derivatives may therefore be described as “prodrugs”. All prodrugs of compounds are included within this disclosure.

Furthermore, all compounds which exist in free base or acid form can be converted to their pharmaceutically acceptable salts by treatment with the appropriate inorganic or organic base or acid by methods known to one skilled in the art. Salts of the compounds can be converted to their free base or acid form by standard techniques.

A synthesis of a dioxaphospholane 2-oxide intermediate A is illustrated below:

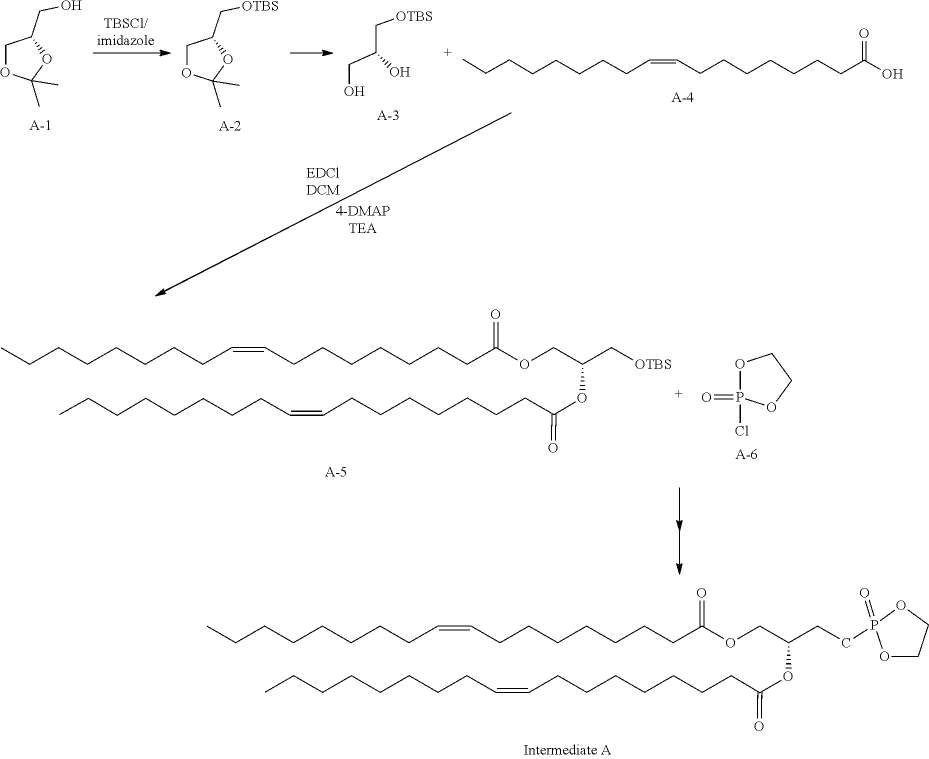

An acetonide protected alcohol (A-1) was protected by reacting with tert-butyldimethylsilyl ether chloride (TBSCl) under basic condition to afford the TBS protected compound (A-2). The TBS protected compound (A-2) underwent deprotection of acetonides to afford a TBS protected diol (A-3) which was coupled with 2 equivalents of acids (A-4) under EDC coupling condition to generate a coupled product (A-5). The TBS group of the coupled product (A-5) was deprotected by TBAF followed by further reacting with 2-chloro-1,3,2-dioxaphospholane 2-oxide (A-6) to afford intermediate A.

A synthesis of an amine intermediate B is illustrated below:

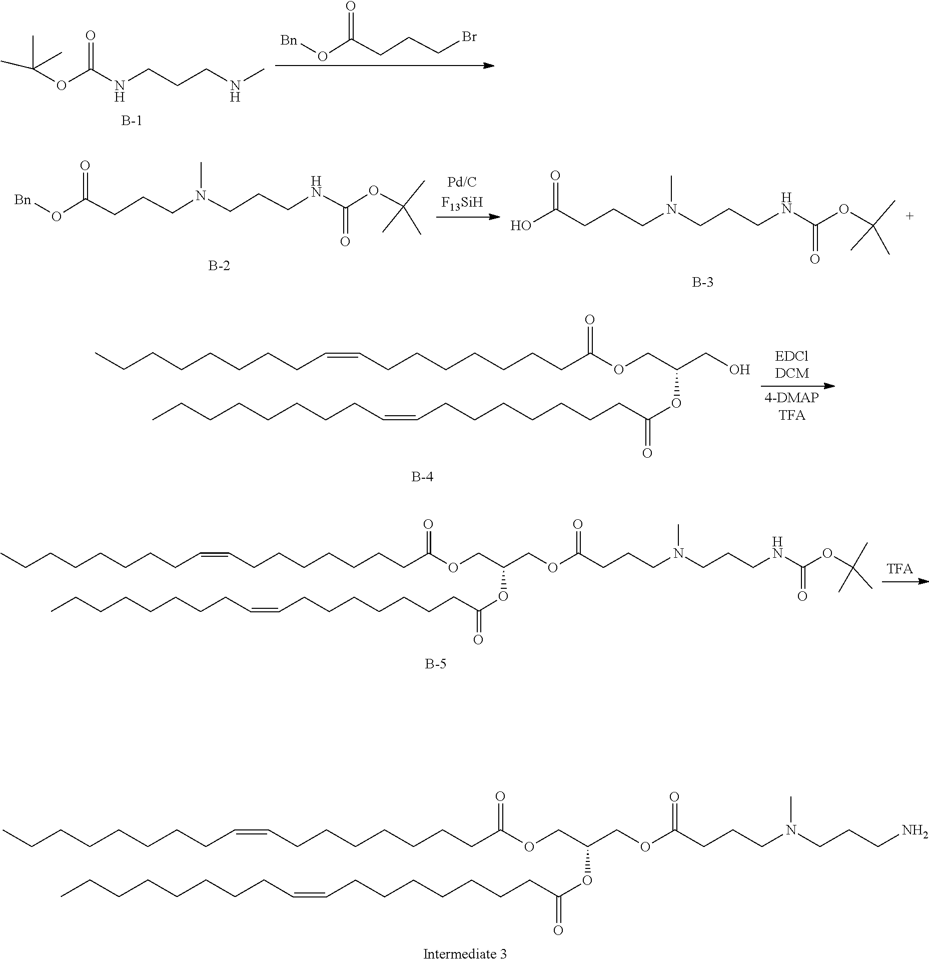

A tert-butyl carbamate (boc) protected amine (B-1) was reacted with benzyl 4-bromobutanoate to afford a compound B-2. A benzyl group of the compound B-2 was removed with palladium on carbon with excess triethylsilane to afford B-3. B-3 was coupled with 1 equivalent of alcohol (B-4) under EDC coupling condition to generate a coupled product (B-5). The boc group of the coupled product (B-5) was deprotected by trifluoroacetic acid (TFA) to afford amine intermediate B.

Compounds of formula (I)—(IIB) were prepared by reacting Intermediate A with Intermediate B or variation of Intermediate B. A synthesis of the representative compound I-1 is illustrated below:

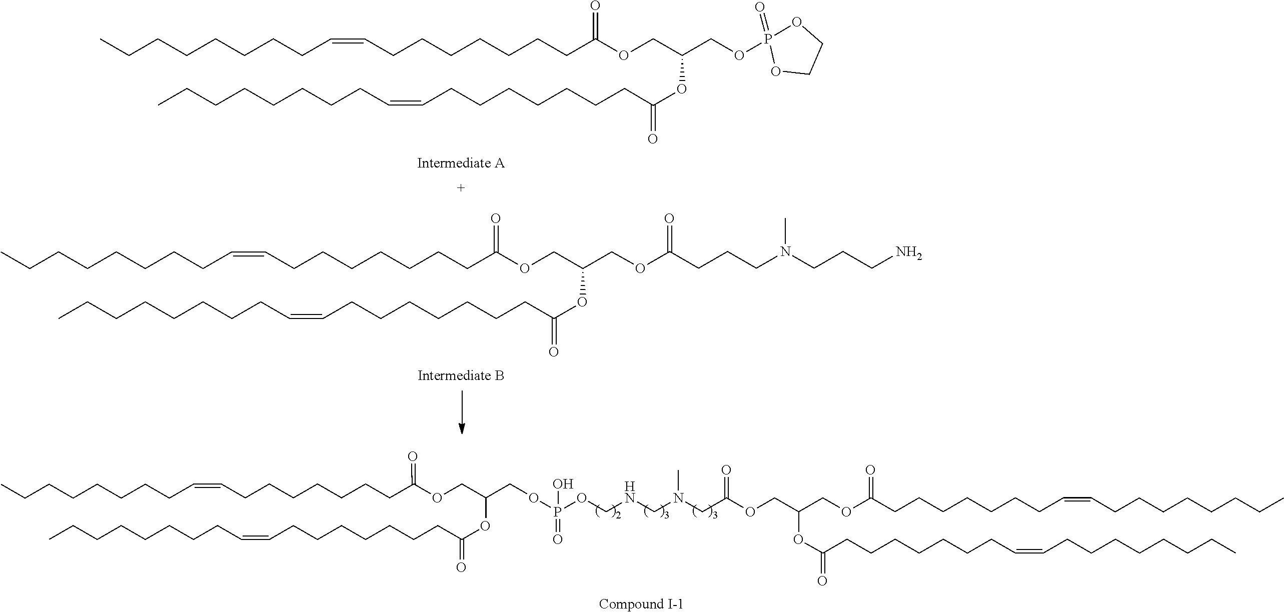

The compound I-2 was prepared by reacting Intermediate A with Intermediate B-1. Intermediate B-1 was similarly prepared accordingly the synthetic scheme of Intermediate B above. A synthesis of the compound I-2 is illustrated below:

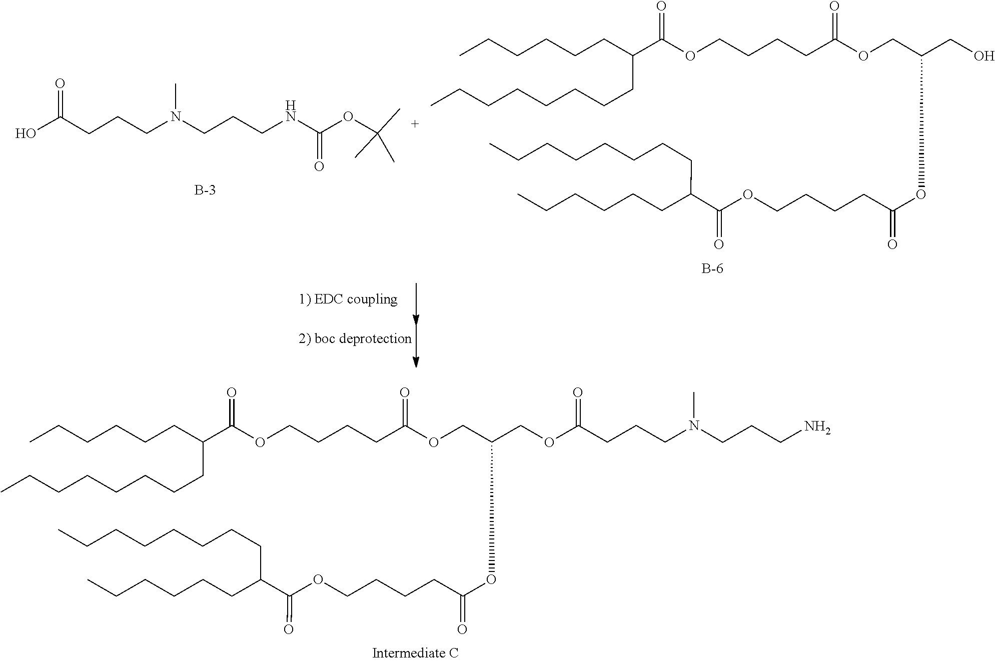

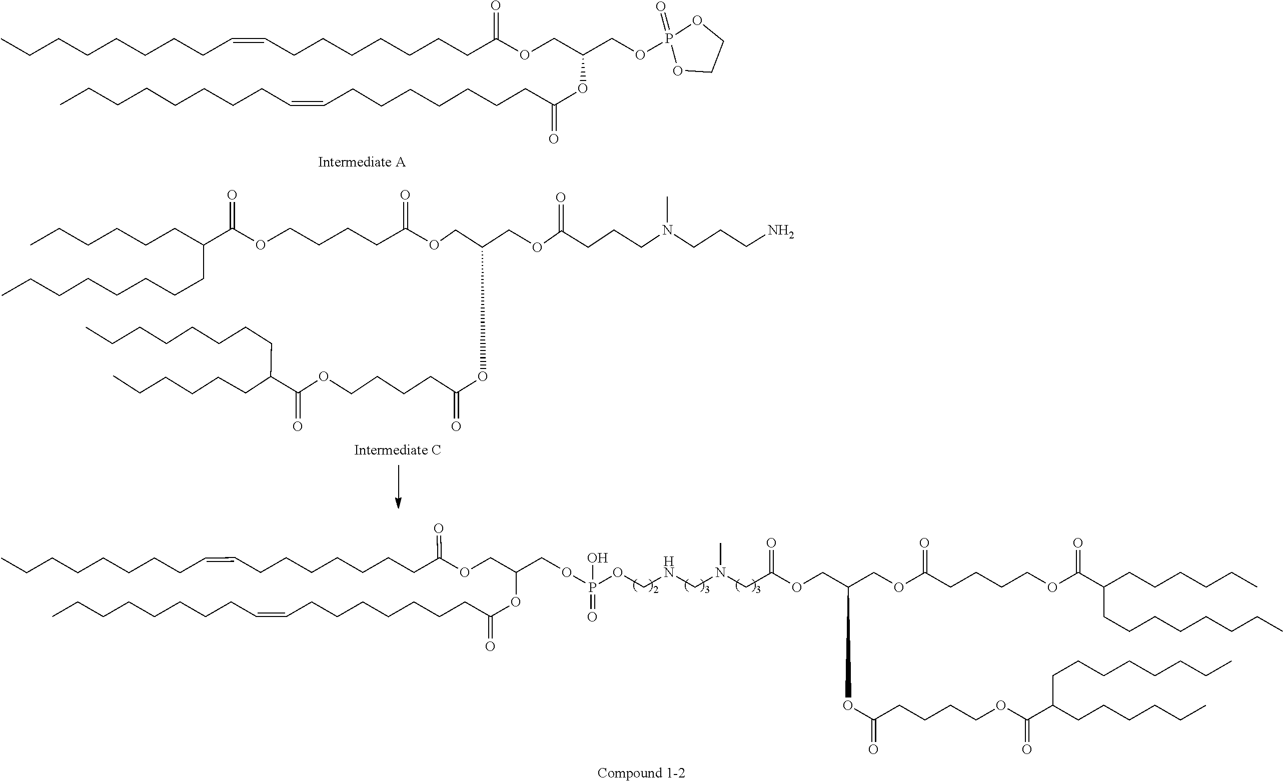

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A compound having the following structure (I): 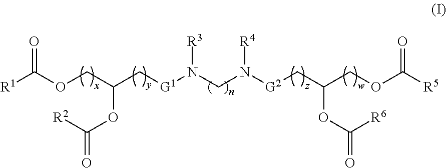
or a pharmaceutically acceptable salt or stereoisomer thereof, wherein:
G¹ and G² are each independently C₁-C₆ alkyl ester, C₁-C₆ alkyl amide, or C₁-C₆ alkyl phosphate, wherein one of G¹ and G² is C₁-C₆ alkyl amide or C₁-C₆ alkyl phosphate,
R¹, R², R⁵, and R⁶ are each independently C₁₂-C₂₄ alkyl, C₁₂-C₂₄ alkylene, or C₁-C₆ alkyl, wherein the C₁-C₆ alkyl is substituted with —OC(═O)R⁷, —NR⁸C(═O)R⁷, or —C(═O)OR⁷;
R³ and R⁴ are each independently H, C₁-C₆ alkyl, or C₁-C₆ alkyl alcohol;
R⁷ is branched C₆-C₂₄ alkyl or C₂-C₁₆ heteroalkyl;
R⁸ is H or C₁-C₆ alkyl;
x and w are each independently an integer ranging from 1 to 4;
n is an integer ranging from 1 to 12; and
y, and z are each independently an integer ranging from 1 to 6.

2. The compound of claim 1, wherein G¹ and G² are each independently C₁-C₆ alkyl ester or C₁-C₆ alkyl phosphate.

3. The compound of any one of claims 1-2, wherein G¹ and G² are each independently C₁-C₄ alkyl ester or C₁-C₄ alkyl phosphate.

4. The compound of any one of claims 1-3, wherein G¹ and G² are each independently C₁-C₃ alkyl ester or C₁-C₃ alkyl phosphate.

5. The compound of any one of claims 1-4, wherein G¹ and G² are each independently C₃ alkyl ester or C₂ alkyl phosphate.

6. The compound of claim 5, having one of the following structures (IA)-(ID): 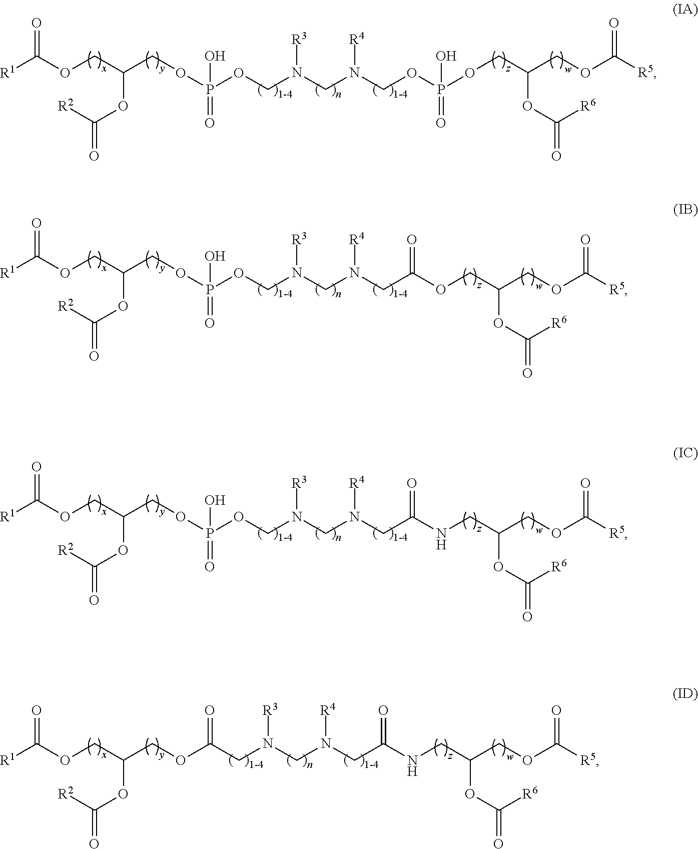
or a pharmaceutically acceptable salt or stereoisomer thereof.

7. The compound of any one of claims 1-6, wherein R¹, R², R⁵, and R⁶ are each independently C₁₂-C₂₄ alkylene or C₁-C₆ alkyl, wherein the C₁-C₆ alkyl is substituted with —OC(═O)R⁷, —NR⁸C(═O)R⁷, or —C(═O)OR⁷.

8. The compound of any one of claims 1-7, wherein R¹, R², R⁵, and R⁶ are each independently C₁₂-C₂₄ alkylene or C₁-C₆ alkyl, wherein the C₁-C₆ alkyl is substituted with —OC(═O)R⁷ or —NR⁸C(═O)R⁷.

9. The compound of any one of claims 1-8, wherein R¹, R², R⁵, and R⁶ are each independently C₁₂-C₂₄ alkylene or C₁-C₆ alkyl, wherein the C₁-C₆ alkyl is substituted with —OC(═O)R⁷.

10. The compound of any one of claims 1-9, wherein R¹, R², R⁵, and R⁶ are each independently C₁₄-C₂₀ alkylene or C₂-C₅ alkyl, wherein the C₂-C₅ alkyl is substituted with —OC(═O)R⁷ or —NR⁸C(═O)R⁷.

11. The compound of any one of claims 1-10, wherein R¹, R², R⁵, and R⁶ are each independently C₁₄-C₂₀ alkylene or C₂-C₅ alkyl, wherein the C₂-C₅ alkyl is substituted with —OC(═O)R⁷.

12. The compound of any one of claims 1-11, wherein R¹, R², R⁵, and R⁶ are each independently C₁₇ alkylene or C₄ alkyl, wherein the C₄ alkyl is substituted with —OC(═O)R⁷.

13. The compound of any one of claims 1-12, wherein R¹ and R² are C₁₇ alkylene and R⁵ and R⁶ are each independently C₁₇ alkylene or C₄ alkyl, wherein the C₄ alkyl is substituted with —OC(═O)R⁷.

14. The compound of any one of claims 1-13, wherein R⁷ has one of the following structures:

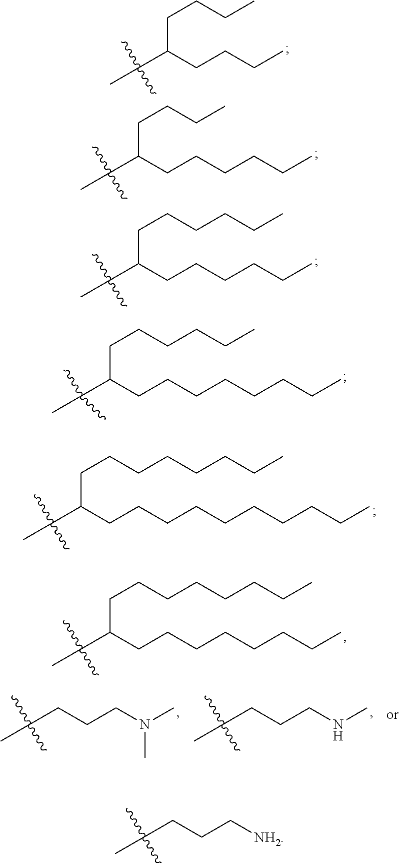

15. The compound of any one of claims 1-14, wherein R⁷ has one of the following structures:

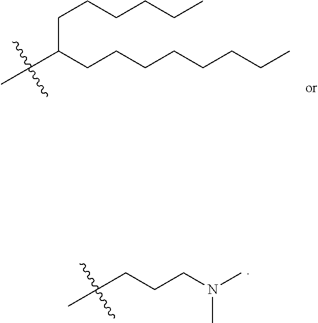

16. The compound of any one of claims 1-15, wherein R⁸ is H or C₁-C₃ alkyl.

17. The compound of any one of claims 1-16, wherein R⁸ is H or methyl.

18. The compound of any one of claims 1-17, wherein R¹, R², R⁵, and R⁶ each independently have one of the following structures:

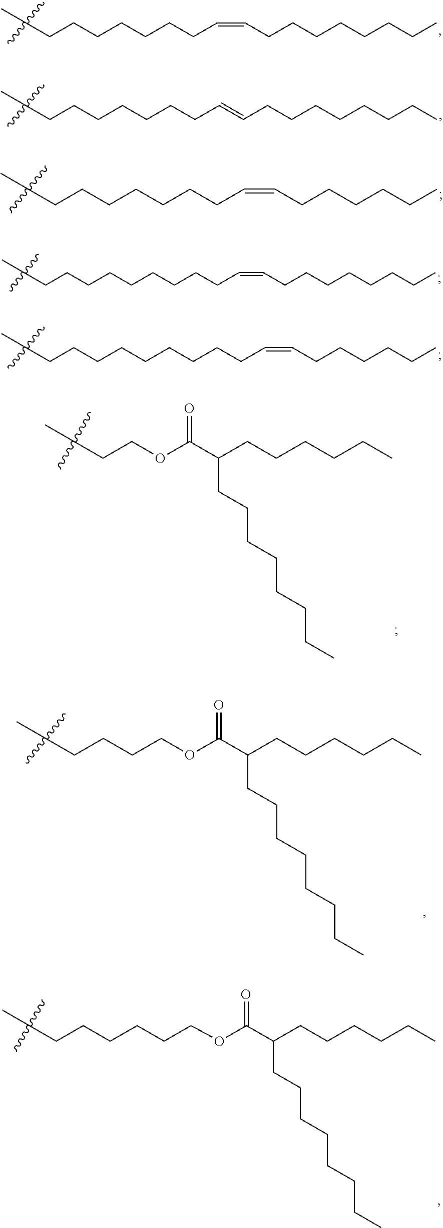

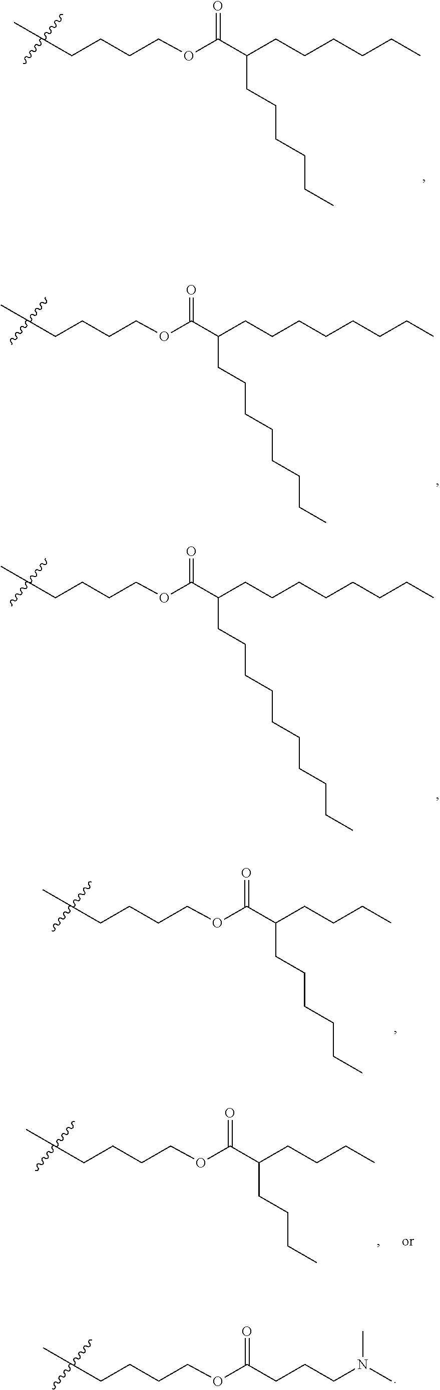 [SMILES: *CCCCOC(=O)C(CCCCCC)CCCCCC ; *CCCCOC(=O)C(CCCCCCCC)CCCCCCCC ; *CCCCOC(=O)C(CCCCCCCC)CCCCCCCCCC ; *CCCCOC(=O)C(CCCC)CCCCCC ; *CCCCOC(=O)C(CCCC)CCCC ; *CCCCOC(=O)CCCN(C)C]

19. The compound of any one of claims 1-18, wherein R³ and R⁴ are each independently H, C₁-C₃ alkyl, or C₁-C₄ alkyl alcohol.

20. The compound of any one of claims 1-19, wherein one of R³ or R⁴ is H and the other one of R³ or R⁴ is C₁-C₃ alkyl, or C₁-C₄ alkyl alcohol.

21. The compound of any one of claims 1-20, wherein one of R³ or R⁴ is H and the other one of R³ or R⁴ is C₁-C₃ alkyl.

22. The compound of any one of claims 1-21, wherein one of R³ or R⁴ is H and the other one of R³ or R⁴ is methyl.

23. The compound of any one of claims 1-22, wherein one of R³ or R⁴ is H and the other one of R³ or R⁴ is C₁-C₄ alkyl alcohol.

24. The compound of any one of claims 1-23, wherein one of R³ or R⁴ is H and the other one of R³ or R⁴ is C₂-C₄ alkyl alcohol.

25. The compound of any one of claims 1-24, wherein n is 3.

26. The compound of any one of claims 1-25, wherein n is 4.

27. The compound of any one of claims 1-26, wherein x and w are each independently an integer of 1.

28. The compound of any one of claims 1-27, wherein x and w are each independently an integer of 2.

29. The compound of any one of claims 1-28, wherein y and z are each independently an integer is 1.

30. The compound of any one of claims 1-29, wherein y and z are each independently an integer is 2.

31. The compound of claim 1, having one of the following structures: 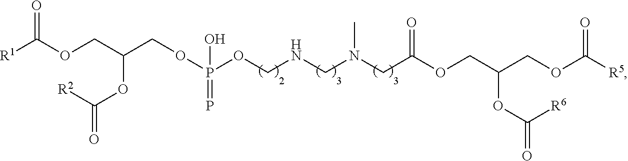
wherein R¹, R², R⁵, and R⁶ are 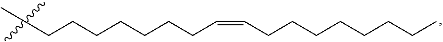
wherein R¹ and R² are 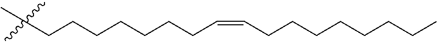 [SMILES: *CCCCCCC/C=C\CCCCCCCC]
and R⁵ and R⁶ are 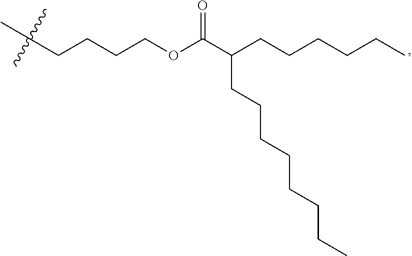
wherein R¹, R², R⁵, and R⁶ are 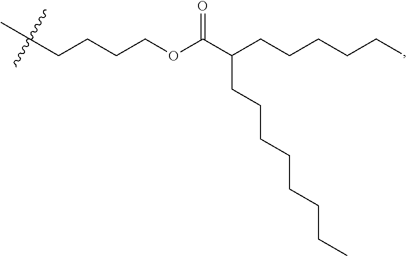
wherein R¹ and R² are 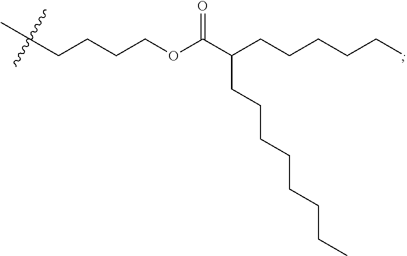
R⁵ is 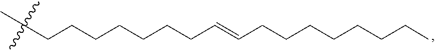 [SMILES: *CCCCCCC/C=C/CCCCCCCC]
and R⁶ is 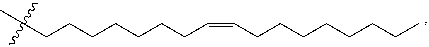 [SMILES: *CCCCCCC/C=C\CCCCCCCC]
or a pharmaceutically acceptable salt or stereoisomer thereof.

32. A pharmaceutical composition comprising the compound of any one of claims 1-31 and a therapeutic agent comprising a nucleic acid.

33. The pharmaceutical composition of claim 32, further comprising a helper lipid, a stabilization lipid, and/or a structural lipid.

34. The pharmaceutical composition of claim 33, wherein the helper lipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and mixtures thereof.

35. The pharmaceutical composition of claim 33, wherein the stabilization lipid is 1-(monomethoxy-polyethyleneglycol)-2,3-dimyristoylglycerol (PEG-DMG), with an average PEG molecular weight of 2000.

36. The pharmaceutical composition of claim 33, wherein the structural lipid is selected from the group consisting of cholesterol, cholesterol derivatives, fecosterol, sitosterol, ergosterol, campesterol, stigmasterol, brassicasterol, tomatidine, ursolic acid, alpha tocopherol, and mixtures thereof.

37. The pharmaceutical composition of claim 32, wherein the nucleic acid is selected from small interfering RNA (siRNA), an asymmetrical interfering RNA (aiRNA), a microRNA (miRNA), a Dicer-substrate RNA (dsRNA), a small hairpin RNA (shRNA), or a messenger RNA (mRNA).

38. The pharmaceutical composition of claim 37, wherein the nucleic acid is mRNA.

39. A method for administering a therapeutic agent comprising a nucleic acid to a patient in need thereof, the method comprising administering the composition of claim 32 to the patient.

40. A lipid nanoparticle comprising the compound of any one of claims 1-31 and a therapeutic agent comprising a nucleic acid.

41. The lipid nanoparticle of claim 40, wherein the therapeutic agent is mRNA.

42. A compound having the following structure (II): 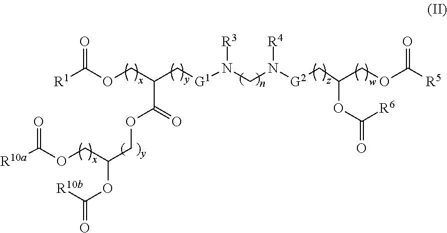
or a pharmaceutically acceptable salt or stereoisomer thereof, wherein:
G¹ is C₁-C₆ alkyl phosphate;
G² is C₁-C₆ alkyl ester, C₁-C₆ alkyl amide, or C₁-C₆ alkyl phosphate;
R¹, R⁵, R⁶, R^{10a}, and R^{10b} are each independently C₁₂-C₂₄ alkyl, C₁₂-C₂₄ alkylene, or C₁-C₆ alkyl, wherein the C₁-C₆ alkyl is substituted with —OC(═O)R⁷, —NR⁸C(═O)R⁷, or —C(═O)OR⁷;
R³ and R⁴ are each independently H, C₁-C₆ alkyl, or C₁-C₆ alkyl alcohol;
R⁷ is branched C₆-C₂₄ alkyl or C₂-C₁₆ heteroalkyl;
R⁸ is H or C₁-C₆ alkyl;
x and w are each independently an integer ranging from 1 to 4;
n is an integer ranging from 1 to 12; and
y, and z are each independently an integer ranging from 1 to 6.

43. The compound of claim 42, having one of the following structures (IIA)-(IIB): 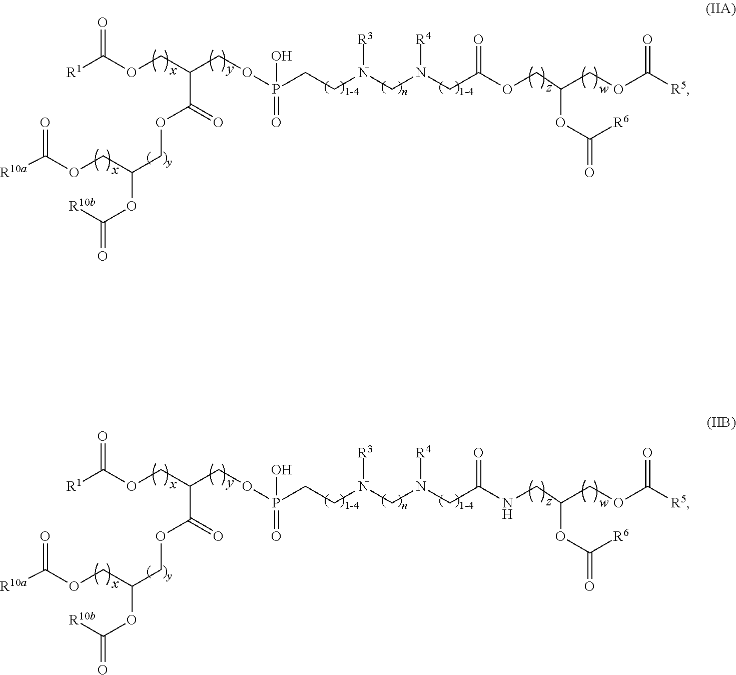
or a pharmaceutically acceptable salt or stereoisomer thereof.

44. The compound of any one of claims 42-43, wherein R¹, R⁵, R⁶, R^{10a}, and R^{10b} are each independently C₁₂-C₂₄ alkylene or C₁-C₆ alkyl, wherein the C₁-C₆ alkyl is substituted with —OC(═O)R⁷, —NR⁸C(═O)R⁷, or —C(═O)OR⁷.

45. The compound of any one of claims 42-44, wherein R¹, R⁵, R⁶, R^{10a}, and R^{10b} are each independently C₁₂-C₂₄ alkylene or C₁-C₆ alkyl, wherein the C₁-C₆ alkyl is substituted with —OC(═O)R⁷ or —NR⁸C(═O)R⁷.

46. The compound of any one of claims 42-45, wherein R¹, R⁵, R⁶, R^{10a}, and R^{10b} are each independently C₁₂-C₂₄ alkylene or C₁-C₆ alkyl, wherein the C₁-C₆ alkyl is substituted with —OC(═O)R⁷.

47. The compound of any one of claims 42-46, wherein R⁷ has one of the following structures:

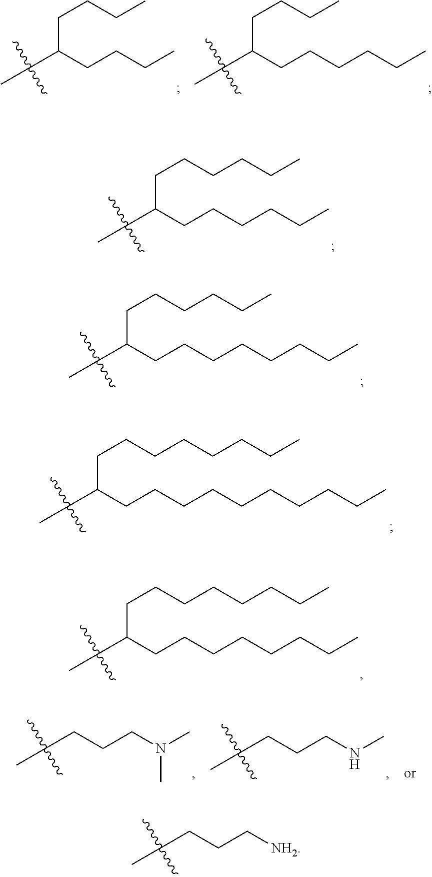

48. The compound of any one of claims 42-47, wherein R⁷ has one of the following structures:

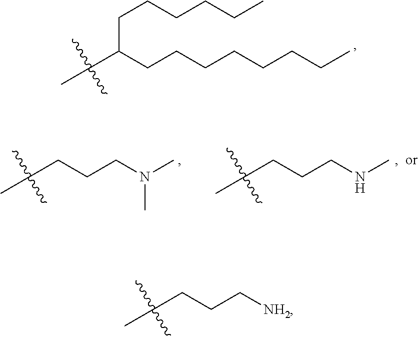

49. The compound of any one of claims 42-48, wherein R⁸ is H or C₁-C₃ alkyl.

50. The compound of any one of claims 42-49, wherein R⁸ is H or methyl.

51. The compound of any one of claims 42-50, wherein R¹, R⁵, R⁶, R^{10a}, and R^{10b} each independently have one of the following structures:

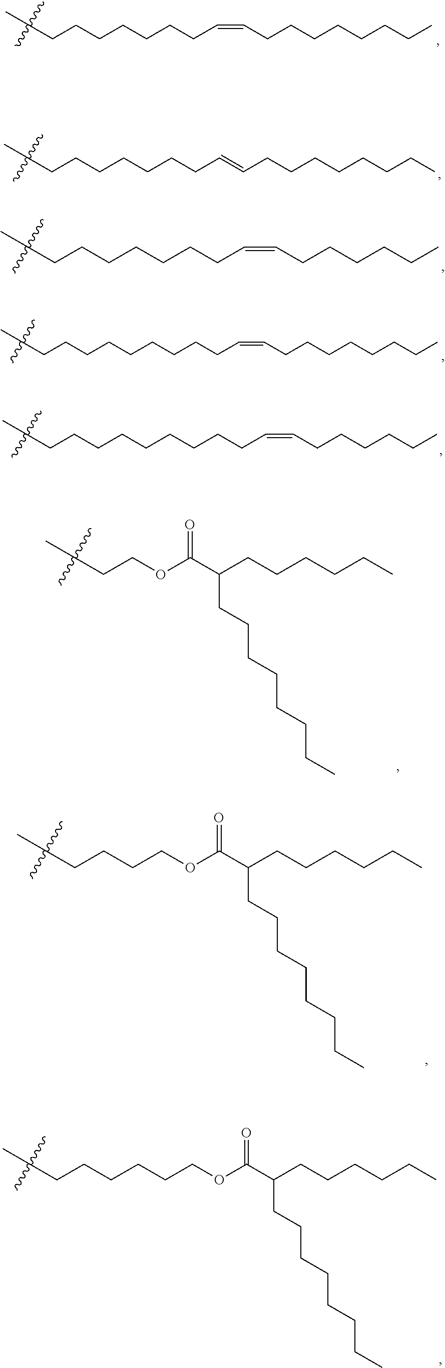

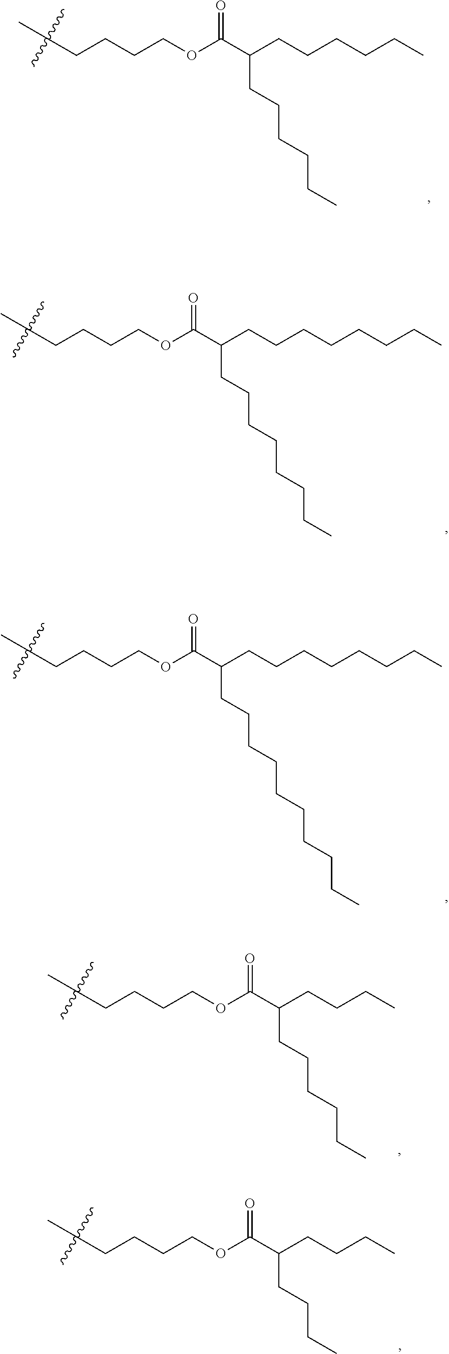 [SMILES: *CCCCOC(=O)C(CCCCCC)CCCCCC ; *CCCCOC(=O)C(CCCCCCCC)CCCCCCCC ; *CCCCOC(=O)C(CCCCCCCC)CCCCCCCCCC ; *CCCCOC(=O)C(CCCC)CCCCCC ; *CCCCOC(=O)C(CCCC)CCCC]

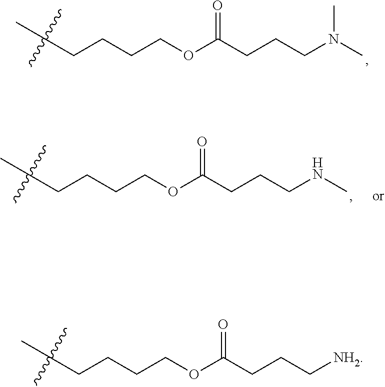 [SMILES: *CCCCOC(=O)CCCN(C)C ; *CCCCOC(=O)CCCNC ; *CCCCOC(=O)CCCN]

52. The compound of any one of claims 42-51, wherein R³ and R⁴ are each independently H, C₁-C₃ alkyl, or C₁-C₄ alkyl alcohol.

53. The compound of any one of claims 42-52, wherein one of R³ or R⁴ is H and the other one of R³ or R⁴ is C₁-C₃ alkyl, or C₁-C₄ alkyl alcohol.

54. The compound of any one of claims 42-53, wherein one of R³ or R⁴ is H and the other one of R³ or R⁴ is C₁-C₃ alkyl.

55. The compound of any one of claims 42-54, wherein one of R³ or R⁴ is H and the other one of R³ or R⁴ is methyl.

56. The compound of any one of claims 42-55, wherein one of R³ or R⁴ is H and the other one of R³ or R⁴ is C₁-C₄ alkyl alcohol.

57. The compound of any one of claims 42-56, wherein one of R³ or R⁴ is H and the other one of R³ or R⁴ is C₂-C₄ alkyl alcohol.

58. The compound of any one of claims 42-57, wherein n is 3.

59. The compound of any one of claims 42-58, wherein n is 4.

60. The compound of any one of claims 42-59, wherein x and w are each independently an integer of 1.

61. The compound of any one of claims 42-60, wherein x and w are each independently an integer of 2.

62. The compound of any one of claims 42-61, wherein y and z are each independently an integer is 1.

63. The compound of any one of claims 42-62, wherein y and z are each independently an integer is 2.

64. The compound of claim 42, having one of the following structures: 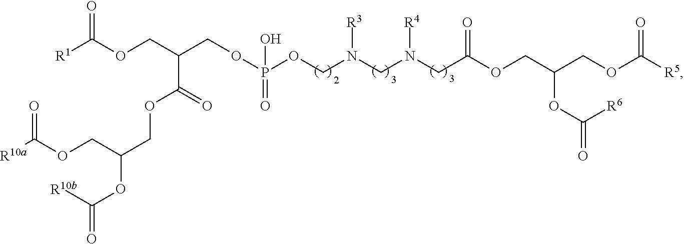
A) wherein R¹ is 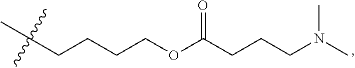 [SMILES: *CCCCOC(=O)CCCN(C)C]
R^{10a} and R^{10b} are 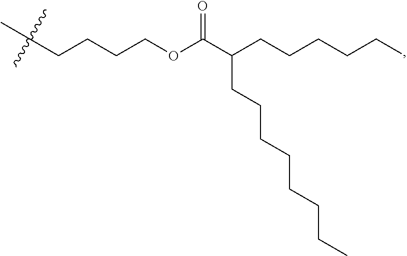
and R⁵ and R⁶ are 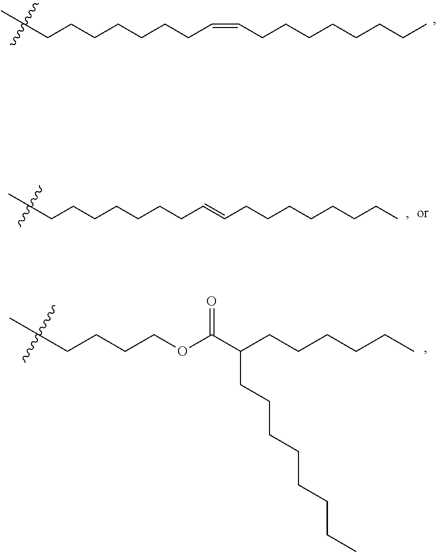 [SMILES: *CCCCCCC/C=C\CCCCCCCC ; *CCCCCCC/C=C/CCCCCCCC ; *CCCCOC(=O)C(CCCCCC)CCCCCCCC]
B) wherein R¹ is 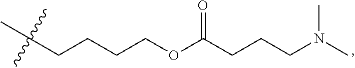 [SMILES: *CCCCOC(=O)CCCN(C)C]
R^{10a} and R^{10b} are 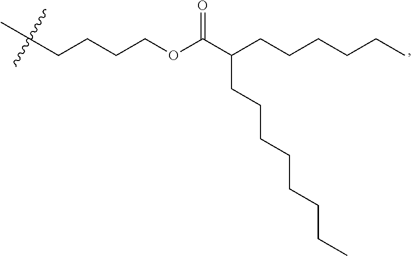 [SMILES: *CCCCOC(=O)C(CCCCCC)CCCCCCCC]
R⁵ is 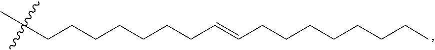 [SMILES: *CCCCCCC/C=C/CCCCCCCC]
and R⁶ is 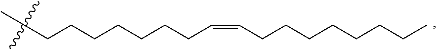 [SMILES: *CCCCCCC/C=C\CCCCCCCC]
C) wherein R¹ is 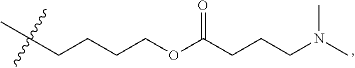 [SMILES: *CCCCOC(=O)CCCN(C)C]
R^{10a} and R^{10b} are 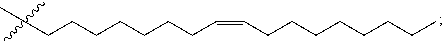 [SMILES: *CCCCCCC/C=C\CCCCCCCC]
and
R⁵ and R⁶ are 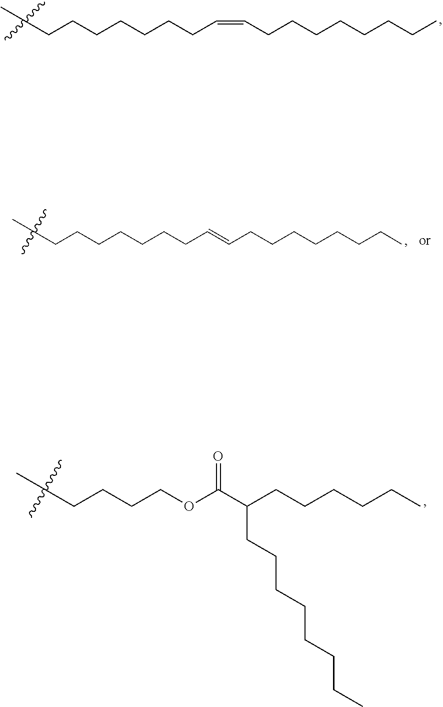 [SMILES: *CCCCCCC/C=C\CCCCCCCC ; *CCCCCCC/C=C/CCCCCCCC ; *CCCCOC(=O)C(CCCCCC)CCCCCCCC]
or a pharmaceutically acceptable salt or stereoisomer thereof.

65. A pharmaceutical composition comprising the compound of any one of claims 42-64 and a therapeutic agent comprising a nucleic acid.

66. The pharmaceutical composition of claim 65, further comprising a helper lipid, a stabilization lipid, and/or a structural lipid.

67. The pharmaceutical composition of claim 66, wherein the helper lipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and mixtures thereof.

68. The pharmaceutical composition of claim 66, wherein the stabilization lipid is 1-(monomethoxy-polyethyleneglycol)-2,3-dimyristoylglycerol (PEG-DMG), with an average PEG molecular weight of 2000.

69. The pharmaceutical composition of claim 66, wherein the structural lipid is selected from the group consisting of cholesterol, cholesterol derivatives, fecosterol, sitosterol, ergosterol, campesterol, stigmasterol, brassicasterol, tomatidine, ursolic acid, alpha tocopherol, and mixtures thereof.

70. The pharmaceutical composition of claim 65, wherein the nucleic acid is selected from small interfering RNA (siRNA), an asymmetrical interfering RNA (aiRNA), a microRNA (miRNA), a Dicer-substrate RNA (dsRNA), a small hairpin RNA (shRNA), or a messenger RNA (mRNA).

71. The pharmaceutical composition of claim 70, wherein the nucleic acid is mRNA.

72. A method for administering a therapeutic agent comprising a nucleic acid to a patient in need thereof, the method comprising administering the composition of any one of claim 65 to the patient.

73. A lipid nanoparticle comprising the compound of any one of claims 42-64 and a therapeutic agent comprising a nucleic acid.

74. The lipid nanoparticle of claim 73, wherein the therapeutic agent is mRNA.
